SEQUENCE LISTING (58 sequences)

SEQ ID NO: 1 - DNA, 24 bp, synthetic construct
  source 1..24: mol_type = other DNA
gtaaacggccacaagttcagcgtg

SEQ ID NO: 2 - DNA, 22 bp, synthetic construct
  source 1..22: mol_type = other DNA
gtgctgcttcatgtggtcgggg

SEQ ID NO: 3 - DNA, 16 bp, synthetic construct
  source 1..16: mol_type = other DNA
atgagcagaccaggag

SEQ ID NO: 4 - DNA, 21 bp, synthetic construct
  source 1..21: mol_type = other DNA
atatgaagacttgttaccagc

SEQ ID NO: 5 - DNA, 20 bp, synthetic construct
  source 1..20: mol_type = other DNA
attgttttggcggaagggga

SEQ ID NO: 6 - DNA, 20 bp, synthetic construct
  source 1..20: mol_type = other DNA
tcaaagctgctaccaccacc

SEQ ID NO: 7 - DNA, 20 bp, synthetic construct
  source 1..20: mol_type = other DNA
aggccatgcacaggttactc

SEQ ID NO: 8 - DNA, 20 bp, synthetic construct
  source 1..20: mol_type = other DNA
aggagtggaccaggacttgt

SEQ ID NO: 9 - DNA, 20 bp, synthetic construct
  source 1..20: mol_type = other DNA
ggaagaagatcggtggtgct

SEQ ID NO: 10 - DNA, 20 bp, synthetic construct
  source 1..20: mol_type = other DNA
aataccaagggcggcaaaga

SEQ ID NO: 11 - DNA, 20 bp, synthetic construct
  source 1..20: mol_type = other DNA
tgaagcagcaggaagtggaa

SEQ ID NO: 12 - DNA, 20 bp, synthetic construct
  source 1..20: mol_type = other DNA
cccatccaaactgaacccca

SEQ ID NO: 13 - DNA, 20 bp, synthetic construct
  source 1..20: mol_type = other DNA
actgccctgacatggattgg

SEQ ID NO: 14 - DNA, 21 bp, synthetic construct
  source 1..21: mol_type = other DNA
ccccattttcgacagagcttc

SEQ ID NO: 15 - DNA, 20 bp, synthetic construct
  source 1..20: mol_type = other DNA
tggtttaggaacagtgcaac

SEQ ID NO: 16 - DNA, 20 bp, synthetic construct
  source 1..20: mol_type = other DNA
ttgcttctcctcttggtgag

SEQ ID NO: 17 - DNA, 20 bp, synthetic construct
  source 1..20: mol_type = other DNA
accagacatttcagctgcct

SEQ ID NO: 18 - DNA, 22 bp, synthetic construct
  source 1..22: mol_type = other DNA
ggctagcccttttctttccaag

SEQ ID NO: 19 - DNA, 20 bp, synthetic construct
  source 1..20: mol_type = other DNA
ttggaggtgtatctgcccac

SEQ ID NO: 20 - DNA, 20 bp, synthetic construct
  source 1..20: mol_type = other DNA
gattccggctacaaccccaa

SEQ ID NO: 21 - DNA, 20 bp, synthetic construct
  source 1..20: mol_type = other DNA
acatgcctaacggtagcctg

SEQ ID NO: 22 - DNA, 20 bp, synthetic construct
  source 1..20: mol_type = other DNA
cagatcgccgtccagaagaa

SEQ ID NO: 23 - DNA, 22 bp, synthetic construct
  source 1..22: mol_type = other DNA
ctgctctctcagtagccaacac

SEQ ID NO: 24 - DNA, 22 bp, synthetic construct
  source 1..22: mol_type = other DNA
cttcctccaatagcagaggttt

SEQ ID NO: 25 - DNA, 32 bp, synthetic construct
  source 1..32: mol_type = other DNA
cgggatccaacggagtagtaaattttctttag

SEQ ID NO: 26 - DNA, 30 bp, synthetic construct
  source 1..30: mol_type = other DNA
aaggcctcataacttagatacaaccccacc

SEQ ID NO: 27 - DNA, 27 bp, synthetic construct
  source 1..27: mol_type = other DNA
cgggatccgtaagcaaattaagggccc

SEQ ID NO: 28 - DNA, 41 bp, synthetic construct
  source 1..41: mol_type = other DNA
tcccccgggtttttttcctataataaaaaataaaaaagaat

SEQ ID NO: 29 - DNA, 27 bp, synthetic construct
  source 1..27: mol_type = other DNA
ctttcaaaggagagattcatgccaaag

SEQ ID NO: 30 - DNA, 27 bp, synthetic construct
  source 1..27: mol_type = other DNA
ctttggcatgaatctgccctttgaaag

SEQ ID NO: 31 - DNA, 30 bp, synthetic construct
  source 1..30: mol_type = other DNA
cataactttgcaagccgctctagctgcttc

SEQ ID NO: 32 - DNA, 30 bp, synthetic construct
  source 1..30: mol_type = other DNA
gaagcagctagaggcgcttgcaaagttatg

SEQ ID NO: 33 - DNA, 28 bp, synthetic construct
  source 1..28: mol_type = other DNA
caactttgctagtaggatggagtgtttc

SEQ ID NO: 34 - DNA, 28 bp, synthetic construct
  source 1..28: mol_type = other DNA
gaaacactccatcgcactagcaaagttg

SEQ ID NO: 35 - DNA, 59 bp, synthetic construct
  source 1..59: mol_type = other DNA
ttacggtattaacaacgaaaaaaaatgtaaaaaaaataaaattgtatctaagttccatg

SEQ ID NO: 36 - DNA, 59 bp, synthetic construct
  source 1..59: mol_type = other DNA
catggaacttagatacaattttattttttttacattttttttcgttgttaataccgtaa

SEQ ID NO: 37 - DNA, 27 bp, synthetic construct
  source 1..27: mol_type = other DNA
aaggcctaacttagatacaaccccacc

SEQ ID NO: 38 - DNA, 27 bp, synthetic construct
  source 1..27: mol_type = other DNA
aaggcctaacttagatacaattttatt

SEQ ID NO: 39 - DNA, 24 bp, synthetic construct
  source 1..24: mol_type = other DNA
cgggatccatgagcagaccaggag

SEQ ID NO: 40 - DNA, 28 bp, synthetic construct
  source 1..28: mol_type = other DNA
aaggcctatatgaagacttgttaccagc

SEQ ID NO: 41 - DNA, 24 bp, synthetic construct
  source 1..24: mol_type = other DNA
actactgcaaataacaactaccaa

SEQ ID NO: 42 - DNA, 25 bp, synthetic construct
  source 1..25: mol_type = other DNA
gtataatatttgtgtaatacacgta

SEQ ID NO: 43 - DNA, 19 bp, synthetic construct
  source 1..19: mol_type = other DNA
aactggacaaggcactagc

SEQ ID NO: 44 - DNA, 20 bp, synthetic construct
  source 1..20: mol_type = other DNA
caccgaagttcatgccagtc

SEQ ID NO: 45 - DNA, 20 bp, synthetic construct
  source 1..20: mol_type = other DNA
tctgcaccatcgtcaaccac

SEQ ID NO: 46 - DNA, 20 bp, synthetic construct
  source 1..20: mol_type = other DNA
aaacccacgtcatgccagtt

SEQ ID NO: 47 - DNA, 27 bp, synthetic construct
  source 1..27: mol_type = other DNA
ggaattcccggtgactggtactgcaat

SEQ ID NO: 48 - DNA, 27 bp, synthetic construct
  source 1..27: mol_type = other DNA
aaggcctccagacttccatccagagcg

SEQ ID NO: 49 - DNA, 25 bp, synthetic construct
  source 1..25: mol_type = other DNA
ggaattcgctggtagcgaatcaatg

SEQ ID NO: 50 - DNA, 23 bp, synthetic construct
  source 1..23: mol_type = other DNA
aaggcctaacatcccttgcctcc

SEQ ID NO: 51 - DNA, 27 bp, synthetic construct
  source 1..27: mol_type = other DNA
ggaattcattggctcgagtgatcgcaa

SEQ ID NO: 52 - DNA, 29 bp, synthetic construct
  source 1..29: mol_type = other DNA
tcccccgggtcagtcttggcctcgtgtac

SEQ ID NO: 53 - DNA, 24 bp, synthetic construct
  source 1..24: mol_type = other DNA
attgctagcttgaaacaagtacaa

SEQ ID NO: 54 - DNA, 24 bp, synthetic construct
  source 1..24: mol_type = other DNA
aaacttgtacttgtttcaagctag

SEQ ID NO: 55 - DNA, 24 bp, synthetic construct
  source 1..24: mol_type = other DNA
attgtgattcaatcaaaatatgag

SEQ ID NO: 56 - DNA, 24 bp, synthetic construct
  source 1..24: mol_type = other DNA
aaacctcatattttgattgaatca

SEQ ID NO: 57 - DNA, 24 bp, synthetic construct
  source 1..24: mol_type = other DNA
gattggatgtggtgagccaagaca

SEQ ID NO: 58 - DNA, 24 bp, synthetic construct
  source 1..24: mol_type = other DNA
aaactgtcttggctcaccacatcc

(19) United States
(12) Patent Application Publication
(10) Pub. No.: US 2024/0052002 A1
(43) Pub. Date: Feb. 15, 2024

(54) TOMATO-DERIVED SIJUL GENE REGULATING PHLOEM DEVELOPMENT AND USE THEREOF

(71) Applicant: POSTECH RESEARCH AND BUSINESS DEVELOPMENT FOUNDATION, Pohang-si (KR)

(72) Inventors: Ho Young NAM, Pohang-si (KR); Il Doo HWANG, Pohang-si (KR)

(21) Appl. No.: 18/365,274

(22) Filed: Aug. 4, 2023

(30) Foreign Application Priority Data
Aug. 11, 2022 (KR) 10-2022-0100433

(51) Int. Cl. C07K 14/415; C12N 15/82

(52) U.S. Cl. CPC C07K 14/415; C12N 15/8213; C12N 15/8226; C12N 15/8218; C12N 2310/20

(57) ABSTRACT

The present invention relates to a composition for enhancing the sink strength of a sink tissue of a plant. The composition can increase the number of phloem cells and phloem transport velocity by suppressing the expression of an SlJUL protein or a gene encoding the SlJUL protein. Therefore, the present invention can be usefully used to increase the productivity and yield of agricultural crops.

### CROSS-REFERENCE TO RELATED APPLICATION

This application claims priority to and the benefit of Korean Patent Application No. 10-2022-0100433, filed on Aug. 11, 2022, the disclosure of which is incorporated herein by reference in its entirety.

### REFERENCE TO AN ELECTRONIC SEQUENCE LISTING

The contents of the electronic sequence listing (IPI20230024US_SEQ.xml; Size: 50.9 K bytes; and Date of Creation: Aug. 4, 2023) is herein incorporated by reference in its entirety. The contents of the electronic sequence listing in no way introduces new matter into the specification.

### BACKGROUND

### 1. Field of the Invention

The present invention relates to a tomato-derived SlJUL gene regulating phloem development and a use thereof.

### 2. Discussion of Related Art

The damage caused by global climate anomalies due to an increase in the concentration of carbon dioxide in the atmosphere is increasing, and accordingly, the 2015 Paris Agreement for regulating carbon dioxide emissions, in which 195 countries participated, shows how important it is to control the concentration of carbon dioxide in the atmosphere for human survival. The process of converting carbon dioxide into carbon compounds through photosynthesis is the core mechanism that constitutes the earth's carbon circulation system and is the primary production process of converting light energy obtained from the sun into organic energy in the Earth's ecosystem. Furthermore, understanding and applying the fundamental principles of the development and growth of a plant, which is an autotroph, is an essential part of the continued survival of mankind, not just the survival of individual humans. Thus, as a strategy for increasing carbon assimilation efficiency and crop productivity in the related art, methods of increasing the activity of enzymes involved in photosynthesis or increasing the expression of transport proteins of a photosynthate have been attempted. In addition to these methods, methods of increasing the transport capacity of phloem, which serve as a transport pathway for photosynthetic products, may also be an important strategy.

Phloem is a living conduit in vascular plants, plays an important function in the development of plants as a pathway for the movement of macromolecules such as photosynthetic products, hormones, mRNA, and proteins, and plays a major role in the development and regulation of a storage organ that stores and uses particularly photosynthetic products. The differentiation of the phloem involves irreversible reprogramming of cells from dividing cells called the (pre)cambium, and in these changes, selective degeneration of organelles including the nucleus, cell wall reconstitution and vacuolar membrane disruption occur through the modulation of signals for transcriptional cascades. After the fate of phloem cells is determined, the nuclei of the initial phloem cells are removed to develop into a sieve tube, and these are combined to form a phloem. As cells lose their ability to be transcribed, post-transcriptional regulatory processes may be required to build phloem networks in plants. However, the mechanism of post-transcriptional regulation, which is the source of phloem differentiation, is not known, and the effect on the formation of a source-sink relationship is not clearly identified.

Accordingly, studies on phloem differentiation have been actively conducted both at home and abroad, and knowledge of phloem differentiation regulation mechanisms is gradually expanding. More specifically, research on the mechanism of phloem differentiation was initiated through the discovery of ALTERED PHLOEM DEVELOPMENT (APL), a gene that regulates phloem development, in 2003 (Bonke et al., 2003), and recently, as regulatory genes, NAC45/86, which is a downstream transcription factor, and NEN1-4, which is involved in enucleation during phloem differentiation, were identified. In addition, major phloem development regulators such as OCTOPUS, BIN2, CVP2, CVL1, BRX, BAM3, and CLE45 were identified. However, no suitable gene for humans to artificially regulate phloem development has been found to date. Accordingly, the present invention intends to propose a method of increasing the productivity of crops by increasing the nutrient storage capacity of the nutrient storage tissue of plants by using a gene capable of regulating phloem development.

### SUMMARY OF THE INVENTION

An aspect is to provide a composition for enhancing the nutrient sink strength of a nutrient sink tissue of a plant, containing an expression inhibitor of an SlJUL protein or a gene encoding the SlJUL protein,
in which the expression inhibitor is one or more selected from the group consisting of (a) a virus-induced gene silencing (VIGS) vector containing an SlJUL protein or a gene encoding the SlJUL protein;
(b) a vector containing an SlJUL mutant protein or SlJUL mutant gene; and
(c) a CRISPR/Cas9 vector that edits an SlJUL protein or a gene encoding the SlJUL protein.

Another aspect is to provide a method for enhancing the sink strength of a sink tissue of a plant, the method including treating a plant body with the composition.

Still another aspect is to provide a plant body with enhanced nutrient sink strength of a nutrient sink tissue by the method.

The present invention provides a composition for enhancing the nutrient sink strength of a nutrient sink tissue of a plant, containing an expression inhibitor of an SlJUL protein or a gene encoding the SlJUL protein,
in which the expression inhibitor is one or more selected from the group consisting of (a) a virus-induced gene silencing (VIGS) vector containing an SlJUL protein or a gene encoding the SlJUL protein;
(b) a vector containing an SlJUL mutant protein or SlJUL mutant gene; and
(c) a CRISPR/Cas9 vector that edits an SlJUL protein or a gene encoding the SlJUL protein.

In the present invention, when one or more vectors selected from the group consisting of the (a) vector, (b) vector and (c) vector are used, by suppressing the expression of an SlJUL protein or a gene encoding the SlJUL protein, the sink strength of a sink tissue of a plant may be enhanced, and the productivity and yield of the plant may be increased.

In one embodiment of the present invention, the nutrient sink tissue of the plant may be one or more selected from the group consisting of a seed, a fruit, a flower, a root and a tuber, and may be specifically a fruit.

In one embodiment of the present invention, the SlJUL protein is an orthologue of AtJUL1, which functions as a negative regulator of phloem development in Arabidopsis thaliana, and may suppress the expression of SUPPRESSOR OF MAX21-LIKES (SMXL5) by binding to a 5′ untranslated region (5′UTR) of SMXL5 mRNA to form a RNA G-quadruplex, and may function as a negative regulator in the phloem development of tomatoes.

In one embodiment of the present invention, the expression inhibitor of the SlJUL protein or the gene encoding the SlJUL protein may increase the number of phloem cells and phloem transport capacity by suppressing the expression of SlJUL, which functions as a negative regulator in the phloem development of tomatoes. Therefore, the expression inhibitor may enhance the ability of a plant to store nutrients and increase the productivity of the plant.

In one embodiment of the present invention, the expression inhibitor may be a VIGS vector, a vector including a mutant protein or gene, a RNAi vector or a CRISPR/Cas9 vector, and may be specifically a VIGS vector.

In one embodiment of the present invention, the (a) vector may include an SlJUL protein or a gene encoding the SlJUL protein to suppress the expression of the SlJUL protein or the gene encoding the SlJUL protein through virus-induced gene silencing (VIGS) and may increase the sink strength and productivity of a plant.

The term “VIGS” refers to a phenomenon in which when a foreign gene is introduced into a viral vector and inoculated into a plant body, the expression of the introduced gene and an endogenous gene homologous to the introduced gene is suppressed by a mechanism similar to that of post-transcriptional gene silencing.

In one embodiment of the present invention, a viral vector used for VIGS may be a tobacco rattle virus (TRV) vector, cucumber mosaic virus (CMV), and potato virus X (PVX), and the (a) vector may be a TRV-SlJUL recombinant vector in which the SlJUL gene is introduced into TRV.

The term “vector” refers to a means for transferring and expressing a foreign gene in a target cell and may be independently reproduced in a host cell while replicating DNA.

In one embodiment of the present invention, the vector may be a plasmid, a Ti-plasmid, a cosmid, an artificial chromosome, a liposome, a binary vector, a double-stranded plant viral vector (for example, CaMV), a single-stranded viral vector or an incomplete plant viral vector.

In one embodiment of the present invention, the (b) vector may include an SlJUL mutant protein or an SlJUL mutant gene. The (b) vector may increase the number of phloem cells and phloem transport capacity by expressing the SlJUL mutant protein or the SlJUL mutant gene to act as a dominant-negative of SlJUL and may increase the sink strength and productivity of a plant.

In one embodiment of the present invention, the mutation may occur by the insertion, deletion, or substitution of bases, and maybe a point mutation or frameshift mutation.

In one embodiment of the present invention, the (b) vector may include an SlJUL^{R20/81/151A} protein or SlJUL^{R20/81/151A} gene.

In one embodiment of the present invention, the (c) vector may be a CRISPR/Cas9 vector which edits an SlJUL protein or a gene encoding the SlJUL protein. The (c) vector may include single guide RNA (sgRNA) including CRISPR RNA (crRNA) and transactivating crRNA (tracrRNA), a CRISPR associated protein (Cas9) protein or a gene encoding the Cas9 protein, and an SlJUL protein or a gene encoding the SlJUL protein.

Further, in one embodiment of the present invention, the (c) vector may knockout an SlJUL protein or a gene encoding the SlJUL protein by editing the SlJUL protein or the gene encoding the SlJUL protein.

In one embodiment of the present invention, the composition may increase the expression of one or more genes selected from the group consisting of SlAPL, SlSUT1, SlSUT2, SlSUT4 and SlSWEET1a. The SlAPL gene is a marker gene for the phloem, and the SlSUT1, SlSUT2, SlSUT4 and SlSWEET1a genes are transporter-related genes.

In one embodiment of the present invention, the composition may increase the fruit yield of a plant. Specifically, the composition may increase the total fruit number and total fruit weight of a plant.

In addition, the composition may increase the fruit sugar content of a plant. Specifically, the composition may increase the total sugar amount, total glucose amount and total fructose amount of a plant.

Furthermore, in one embodiment of the present invention, the composition may increase the root growth of a plant. Specifically, the composition may increase the total fresh weight and dry weight of plant roots.

In one embodiment of the present invention, the plant may be selected from the group consisting of food crops including rice, wheat, barley, corn, soybean, potato, red bean, oats, and sorghum;
vegetable crops including Arabidopsis thaliana, Chinese cabbage, radish, chili pepper, strawberry, tomato, watermelon, cucumber, cabbage, Korean melon, pumpkin, green onion, onion, and carrot;
industrial crops including ginseng, tobacco, cotton, sesame, sugar cane, sugar beet, perilla, peanut, and rapeseed;
root and tuber crops including sweet potato, Jerusalem artichoke, cassava, and yacon;
fruit trees including apple, pear, jujube, peach, kiwi fruit, grape, citrus fruit, persimmon, plum, apricot, and banana trees;
flowers including roses, gladiolus, gerbera, carnations, chrysanthemum, lilies, and tulips; and
fodder crops including ryegrass, red clover, orchard grass, alfalfa, tall fescue, and perennial ryegrass.

In one embodiment of the present invention, the plant may be vegetable crops including Arabidopsis thaliana, Chinese cabbage, radish, chili pepper, strawberry, tomato, watermelon, cucumber, cabbage, Korean melon, pumpkin, green onion, onion, and carrot, and may be specifically tomato.

The present invention provides a method for enhancing the sink strength of a sink tissue of a plant, the method including treating a plant body with the composition.

In the present invention, the composition can increase the number of phloem cells and phloem transport velocity by suppressing the expression of an SlJUL protein or a gene encoding the SlJUL protein. Therefore, by the method, the ability of a plant to store nutrients may be enhanced, and the productivity of the plant may be increased.

The term “plant body” refers to the type of body that plants have, and may include plant cells, plant tissues, plant seeds, and the like.

In one embodiment of the present invention, the “treating of the plant body with the composition” means introducing DNA into the plant or transforming the plant. In the present invention, the transformation may be appropriately selected and used by a person skilled in the art according to known methods and may be selected from known calcium/polyethylene glycol methods for protoplasts, electroporation of protoplasts, methods of microinjection into protoplasts, methods using Agrobacterium, (DNA- or RNA-coated) particle bombardment methods of various plant elements, infections by viruses, and the like.

The present invention provides a plant body with enhanced sink strength of a sink tissue of a plant by the method.

### BRIEF DESCRIPTION OF THE DRAWINGS

The above and other objects, features, and advantages of the present invention will become more apparent to those of ordinary skill in the art by describing in detail exemplary embodiments thereof with reference to the accompanying drawings, in which:

FIG. 1 is a view illustrating the amino acid sequences of SlJUL and AtJUL1. Conserved ZnF domains are underlined and conserved residues are highlighted in different colors. Conserved arginine is required for RNA binding and cysteine can stabilize the zinc-finger structure;

FIG. 2 is a set of views illustrating the subcellular localization of SlJUL and SlJUL^{R20/81/151A} confirmed by GFP signals in Arabidopsis protoplasts. Chlorophyll and DAPI were used as indicators for the cytoplasm and nucleus, respectively, and their locations were observed with a confocal laser scanning microscope;

FIG. 3A illustrates GFP signals measured according to the concentration of SlJUL by fusing GFP to SlSMXL5 5′UTR;

FIG. 3B illustrates GFP signals measured according to the concentration of SlJUL^{R20/81/151A} by fusing GFP to SlSMXL5 5′UTR;

FIG. 4A illustrates luciferase activity measured by fusing luciferase (LUC) to SlSMXL5 5′UTR, and then treating with SlJUL or SlJUL^{R20/81/151A};

FIG. 4B illustrates luciferase activity measured by fusing luciferase (LUC) to mSlSMXL5 5′UTR, and then treating with SlJUL or SlJUL^{R20/81/151A};

FIG. 5 illustrates the expression levels of SlJUL measured in plant organs by qRT-PCR. The expression level was normalized to the expression level of the GAPDH reference gene;

FIG. 6A is a set of views illustrating GUS signals by an SlJUL promoter in the transverse section of immature green fruit and the longitudinal section of ripe red fruit;

FIG. 6B is a set of views illustrating GUS signals in the vascular bundle structure of the cross-section of the anther. Black arrows indicate xylem;

FIG. 6C illustrates the GUS signal in the germinal root, pedicel, stamen, style, sepals, and fruit of a germinated seed;

FIG. 7 illustrates tomatoes in which SlJUL was knocked down with recombinant TRV and the expression levels of SlJUL after 30 days of flowering;

FIG. 8 illustrates the peduncle cross-sections and phloem cell numbers of TRV-GFP (control) and TRV-SlJUL plants. IP means internal phloem, EP means external phloem, C means cambium, and X means xylem;

FIG. 9 illustrates the expression levels of a phloem marker gene SlAPL, a cambium marker gene SlTDR and a xylem marker gene SlIRX3 in TRV-GFP (control) and TRV-SlJUL plants;

FIG. 10A illustrates a schematic view of a binary vector and an sgRNA target, which are capable of inducing the mutation of the SlJUL gene using CRISPR-Cas9;

FIG. 10B illustrates a schematic view of a binary vector which is capable of inducing the mutation of the SlJUL gene using CRISPR-Cas9;

FIG. 11A illustrates the peduncle cross-sections of WT and sljul-Cas9 plants and the expression levels of the phloem marker gene SlAPL;

FIG. 11B illustrates the peduncle cross-sections of WT and sljul-d4-Cas9 plants;

FIG. 12 illustrates the expression levels of the cambium marker gene SlTDR and the xylem marker gene SlIRX3 in WT and sljul-Cas9 plants;

FIG. 13 illustrates the peduncle cross-sections and phloem cell numbers of WT and SlJUL^{R20/81/151A} plants;

FIG. 14 illustrates the expression levels of the phloem marker gene SlAPL, the cambium marker gene SlTDR, and the xylem marker gene SlIRX3 in WT and SlJUL^{R20/81/151A} plants;

FIG. 15 illustrates the pedicel cross-sections and phloem cell numbers of TRV-GFP, TRV-SlJUL, and TRV-SlJUL/TRV-SlSMXL5 plants;

FIG. 16 illustrates the leaf numbers, leaf areas, stem diameters, flower numbers, peduncle lengths, peduncle diameters, leaf photosynthesis efficiencies, and CO₂ assimilation rates of TRV-GFP (control) and TRV-SlJUL plants;

FIG. 17 illustrates the leaf numbers, leaf areas, stem diameters, flower numbers, peduncle lengths, peduncle diameters, leaf photosynthesis efficiencies, and CO₂ assimilation rates of WT and SlJUL^{R20/81/151A} plants;

FIG. 18 illustrates the leaf numbers, leaf areas, stem diameters, flower numbers, peduncle lengths, peduncle diameters, leaf photosynthesis efficiencies, and CO₂ assimilation rates of WT and sljul-Cas9 plants;

FIG. 19A illustrates the petiole cross-sections of TRV-GFP (control) and TRV-SlJUL plants. Black arrows indicate phloem;

FIG. 19B illustrates the petiole cross-sections of WT and SlJUL^{R20/81/151A} plants. Black arrows indicate phloem;

FIG. 19B illustrates the petiole cross-sections of WT and sljul-Cas9 plants. Black arrows indicate phloem;

FIG. 20A illustrates UV fluorescence signals measured 10 minutes after esculin loading in TRV-SlJUL plants;

FIG. 20B illustrates UV fluorescence signals measured 10 minutes after esculin loading in SlJUL^{R20/81/151A} plants;

FIG. 20C illustrates UV fluorescence signals measured 10 minutes after esculin loading in sljul-Cas9 plants;

FIG. 20D illustrates UV fluorescence signals measured 10 minutes after esculin loading in sljul-d4-Cas9 plants;

FIG. 21A illustrates a schematic view of the experiment for measuring pixel intensity changes at the same fixed point in the midrib and esculin transport monitoring results per time interval. It can be confirmed that pixel intensity was measured within 1 mm at a distance of 1.5 cm along the midrib from the esculin-treated position, and monitoring results showed that TRV-SlJUL plants transported faster in a basipetal manner in the midrib than control TRV-GFP plants;

FIG. 21B illustrates UV fluorescence signals measured 10, 20, 30, 40, and 50 minutes after treating abraded leaf lamina on both sides of the midrib with 10 μl of an esculin dye (5 mg/ml) solution. Circles indicate esculin loading sites and bars indicate esculin measurement ranges;

FIG. 21C illustrates estimated esculin export rates through the midrib per unit time;

FIG. 22A illustrates the expression levels of major genes encoding sucrose transporters in the source leaves of TRV-GFP (control) and TRV-SlJUL plants;

FIG. 22B illustrates the expression levels of major genes encoding sucrose transporters in the source leaves of WT and SlJUL^{R20/81/151A} plants;

FIG. 22C illustrates the expression levels of major genes encoding sucrose transporters in the source leaves of WT and sljul-Cas9 plants;

FIG. 23A illustrates longitudinal sections of the peduncle of TRV-GFP (control) and TRV-SlJUL plants at 30 days post-anthesis (dpa);

FIG. 23B illustrates the length and diameter of the sieve tube of TRV-GFP (control) and TRV-SlJUL plants;

FIG. 24 illustrates representative images and average fruit numbers of TRV-GFP (control), TRV-SlJUL and TRV-SlSMXL5/TRV-SlJUL plants, and the average diameter and total fruit weight per plant of red ripe fruits;

FIG. 25 illustrates abortive flowers and fruits in the peduncle of TRV-GFP (control) and TRV-SlJUL plants. Green arrows indicate abortive flowers, and yellow arrows indicate flowers that become fruits;

FIG. 26 illustrates total sugar, glucose, and fructose levels of TRV-GFP (control) and TRV-SlJUL plants. Sugar levels were measured in five representative red ripe fruits of each plant, separation parameters and sugar quantification were performed using a Dionex Ultimate 3000-series high performance liquid chromatograph (Thermo Fisher Scientific equipped with a Sugar-Pak column (Waters) and a Shodex RI-101 detector (Shodex);

FIG. 27 illustrates the representative images and total root fresh weight and dry weight levels of TRV-GFP (control) and TRV-SlJUL plants;

FIG. 28 illustrates representative images and average fruit numbers of WT and SlJUL^{R20/81/151A} plants, and the average diameter and total fruit weight per plant of red ripe fruits;

FIG. 29A illustrates representative images and average fruit numbers of WT and sljul-Cas9 plants, and the average diameter and total fruit weight per plant of red ripe fruits;

FIG. 29B illustrates representative images and average fruit numbers of WT and sljul-d4-Cas9 plants;

FIG. 30 illustrates the trade-off between fruit number and sink strength in fruit size and weight. Two to three months after germination, all but 10 fruits were removed from the vine, and the fruit phenotype was scored at the red ripe stage;

FIG. 31 illustrates representative images of 10 red ripe fruits of TRV-GFP (control) and TRV-SlJUL plants, and the mean diameter and weight of the fruits;

FIG. 32A illustrates a schematic view illustrating the correlation between phloem development, photoassimilate distribution and productivity. The thickness of the blue line and arrow represents phloem transport velocity, and the red arrows indicate phloem; and

FIG. 32B illustrates gene expression, phloem cell number, transport capacity and fruit production in WT, TRV-SlJUL, SlJUL^{R20/81/151A} and sljul-Cas9 plants.

### DETAILED DESCRIPTION OF EXEMPLARY EMBODIMENTS

Hereinafter, the present invention will be described in more detail through Examples. However, these Examples are provided only for exemplarily describing the present invention, and the scope of the present invention is not limited by these Examples.

Experimental Example 1: Materials and Methods

1-1. Plant Materials and Growth Conditions

Seeds of tomato cultivar Micro-Tom were provided by Professor Do-il Choi, Seoul National University, Republic of Korea. All seeds were treated with light at an intensity of 1200 μmols⁻¹m⁻² under long-day conditions (16-hour light treatment/8-hour dark treatment) in a medium (pH 5.7) containing vitamins (Duchefa), 3% sucrose (Duchefa), 0.5% 2-(N-morpholino)ethanesulfonic acid (MES, Sigma-Aldrich) and 0.8% phytoagar (Sigma-Aldrich) and containing half-strength Murashige and Skoog salts, and were germinated at 24° C. At 10 days after sowing (DAS), seedlings were transplanted into potted plants and grown under long-day conditions. Arabidopsis thaliana ecotype Col-0 grown under short-day conditions (8-hour light treatment/16-hour dark treatment) was used for protoplast experiments.

1-2. Plasmid Construction and Tomato Gene Modification

For virus-induced gene silencing (VIGS) analysis, cDNA fragments of off-target-free SlJUL (Solyc08g067180.3.1;214bp) and SlSMXL5 (Solyc07g018070.3.1;549bp) (https://www.zhaolab.org/pssRNAit/) were amplified using a cDNA template derived from the Micro-Tom tomato, and cloned into a pTRV2 vector (pYL156, Addgene plasmid # 148969; http://n2t.net/addgene:148969). For a protoplast reporter assay, the 5′UTR of SlSMXL5 (336 bp) was cloned into a plant expression vector containing GFP or LUC (35S:SlSMXL5 5′UTR-GFP, 35S:SlSMXL5 5′UTR-LUC and 35S:mSlSMXL5 5′UTR-LUC), and the full-length coding sequence (CDS) of SlJUL (513 bp) was cloned into a plant expression vector containing a hemagglutinin (HA) tag (35S:SlJUL::HA). For the preparation of SlJUL^{R20/81/151A}, a point mutation (R20(AGA)(58,59,60)->A(GCA), R81(CGC)(241,241,243)->A(GCC) and R151(AGG)(451,452,453)->A(GCG)) of SlJUL and a point mutation (mSlSMXL5 5′UTR) of SlSMXL5 5′UTR were prepared using a QuikChange Site-Directed Mutagenesis kit (Stratagene California).

To analyze the spatial expression pattern of SlJUL, a sequence upstream 2.0 kbp of a translation initiation site was amplified with Micro-Tom tomato genomic DNA and cloned into pCAMBIA1303 after isolation using the CTAB method (pSlJUL:GUS-GFP). The full-length coding sequence of SlJUL containing point mutations was introduced into a pBI121 binary vector containing a CaMV35S promoter (Cauliflower mosaic virus) and a GUS fusion sequence to form a 35S:SlJUL^{R20/81/151A}::GUS construct.

To generate a CRISPR knockout, sgRNA was designed using the CRISPR-P 2.0 tool (Liu et al., 2017) and used to construct CRISPR vectors. All T-DNA constructs are based on Gateway-compatible pEn-C1.1 (HolgerPuchta, Addgene plasmid #61479; http://n2t.net/addgene:61479) and pDe-CAS9 (Holger Puchta, Addgene plasmid#61433; http://n2t.net/addgene:61433) plasmids. A destination vector pDe-CAS9 expresses Cas9 driven by a PcUbi4-2 promoter [the ubiquitous promoter of parsley (Petroselinum crispum Miller)] and includes the small subunit termination sequence of the RIBULOSE-1,5-BISPHOSPHATE CARBOXYLASE (RBCS3A, pea3A) gene of pea (Pisum sativum L.). A spacer sequence (20 bp) was introduced into an entry vector in the form of an annealed oligonucleotide using a classical cloning method of cutting sequences using BbsI (New England Biolabs). A customized RNA chimera is driven by an Arabidopsis U6-26 promoter. Two programmed sgRNA cassettes were integrated into the destination vector to simultaneously target two different locations (5′UTR and 3′UTR) in SlJUL. The first chimera was constructed using Bsu36I and MluI (New England Biolabs) and the second chimera was constructed using the Gateway LR reaction (Thermo Fischer Scientific) as previously described.

sgRNA targeting between ZnF motif 1 and 2 sequences in SlJUL was designed to generate another CRISPR knockout allele. The T-DNA construct used here was based on a pHAtC (Jinsu Kim, Addgene plasmid #78098; https://www.addgene.org/78098) plasmid. pHAtC expresses Cas9 driven by a 35S promoter, and a customized RNA chimera is driven by an Arabidopsis U6-26 promoter. A spacer sequence (20 bp) was introduced into a plant transformation vector in the form of an annealed oligonucleotide using a classical cloning method of cutting sequences using AarI (Thermo Fischer Scientific). A customized RNA chimera is driven by an Arabidopsis U6-26 promoter.

The final binary plasmids were introduced into the cotyledons explants of 10 DAS seedlings (tomato cultivar Micro-Tom) using Agrobacterium tumefaciens (strain EHA105)-mediated transformation. Tomato transformants were selected in BASTA (1 mg/L; Bayer Crop Science) or hygromycin (5 mg/L; Duchefa). T2 generation of the transgenic 35S:SlJUL^{R20/81/151A} and sljul-Cas9 lines was used for further studies. All the primers used in this study are detailed in the following Table 1.

TABLE 1

| Experiment | Name | Sequence (5′-3′) |
|---|---|---|
| RT-PCR | GFP-RT_F | GTAAACGGCCACAAGTTCAGCGTG |
| | | (SEQ ID NO: 1) |
| | GFP-RT_R | GTGCTGCTTCATGTGGTCGGGG (SEQ |
| | | ID NO: 2) |
| | SlJUL-RT_F | ATGAGCAGACCAGGAG (SEQ ID NO: 3) |
| | SlJUL-RT_R | ATATGAAGACTTGTTACCAGC (SEQ ID |
| | | NO: 4) |
| qRT-PCR | SlJUL-VIGS-qRT_F | ATTGTTTTGGCGGAAGGGGA (SEQ ID |
| | | NO: 5) |
| | SlJUL-VIGS-qRT_R | TCAAAGCTGCTACCACCACC (SEQ ID |
| | | NO: 6) |
| | SlSMXL5-Sl07g018070- | AGGCCATGCACAGGTTACTC (SEQ ID |
| | qRT_F | NO: 7) |
| | SlSMXL5-Sl07g018070- | AGGAGTGGACCAGGACTTGT (SEQ ID |
| | qRT_R | NO: 8) |
| | SlSUT1-Sl11g017010- | GGAAGAAGATCGGTGGTGCT (SEQ ID |
| | qRT_F | NO: 9) |
| | SlSUT1-Sl11g017010-qRT_R | AATACCAAGGGCGGCAAAGA (SEQ ID |
| | | NO: 10) |
| | SlSUT2-Sl05g007190-qRT_F | TGAAGCAGCAGGAAGTGGAA (SEQ ID |
| | | NO: 11) |
| | SlSUT2-Sl05g007190-qRT_R | CCCATCCAAACTGAACCCCA (SEQ ID |
| | | NO: 12) |
| | SlSUT4-Sl04g076960-qRT_F | ACTGCCCTGACATGGATTGG (SEQ ID |
| | | NO: 13) |
| | SlSUT4-Sl04g076960-qRT_R | CCCCATTTTCGACAGAGCTTC (SEQ ID |
| | | NO: 14) |
| | SlSWEET1a-Sl04g064610- | TGGTTTAGGAACAGTGCAAC (SEQ ID |
| | qRT_F | NO: 15) |
| | SlSWEET1a-Sl04g064610- | TTGCTTCTCCTCTTGGTGAG (SEQ ID |
| | qRT_R | NO: 16) |
| | SlAPL-Sl12g017370-qRT_F | ACCAGACATTTCAGCTGCCT (SEQ ID |
| | | NO: 17) |
| | SlAPL-Sl12g017370-qRT_R | GGCTAGCCCTTTTCTTTCCAAG (SEQ |
| | | ID NO: 18) |
| | SlIRX3-Sl07g005840-qRT_F | TTGGAGGTGTATCTGCCCAC (SEQ ID |
| | | NO: 19) |
| | SlIRX3-Sl07g005840-qRT_R | GATTCCGGCTACAACCCCAA (SEQ ID |
| | | NO: 20) |
| | SlTDR-Sl03g093330-qRT_F | ACATGCCTAACGGTAGCCTG (SEQ ID |
| | | NO: 21) |
| | SlTDR-Sl03g093330-qRT_R | CAGATCGCCGTCCAGAAGAA (SEQ ID |
| | | NO: 22) |
| | SlGAPDH-qRT_F | CTGCTCTCTCAGTAGCCAACAC (SEQ |
| | | ID NO: 23) |
| | SlGAPDH-qRT_R | CTTCCTCCAATAGCAGAGGTTT (SEQ |
| | | ID NO: 24) |
| GFP-fused | SlSMXL5-5′UTR-BamH1_F | CGGGATCCAACGGAGTAGTAAATTTTC |
| construction | | TTTAG |
| | | (SEQ ID NO: 25) |
| | SlSMXL5-′UTR + | AAGGCCTCATAACTTAGATACAACCCC |
| | ATG-Stu1_R | ACC |
| | | (SEQ ID NO: 26) |
| GUS-fused | pSlJUL-BamH1_F | CGGGATCCGTAAGCAAATTAAGGGCC |
| construction | | C |
| | | (SEQ ID NO: 27) |
| | pSlJUL-Sma1_R | TCCCCCGGGTTTTTTTCCTATAATAAAA |
| | | AATAAAA |
| | | AAGAAT (SEQ ID NO: 28) |
| Point | SlJUL-R20A_F | CTTTCAAAGGAGAGATTCATGCCAAA |
| mutation | | G |
| | | (SEQ ID NO: 29) |
| | SlJUL-R20A_R | CTTTGGCATGAATCTGCCCTTTGAAAG |
| | | (SEQ ID NO: 30) |
| | SlJUL-R81A_F | CATAACTTTGCAAGCCGCTCTAGCTGC |
| | | TTC (SEQ ID NO: 31) |
| | SlJUL-R81A_R | GAAGCAGCTAGAGGCGCTTGCAAAGT |
| | | TATG (SEQ ID NO: 32) |
| | SlJUL-R151A F | CAACTTTGCTAGTAGGATGGAGTGTTT |
| | | C (SEQ ID NO: 33) |
| | SlJUL-R151A R | GAAACACTCCATCGCACTAGCAAAGT |
| | | TG (SEQ ID NO: 34) |
| | SlSMXL5_5′UTR_SDM_F | TTACGGTATTAACAACGAAAAAAAAT |
| | | GTAAAAAA |
| | | AATAAAATTGTATCTAAGTTCCATG |
| | | (SEQ ID NO: 35) |
| | SlSMXL5_5′UTR_SDM_R | CATGGAACTTAGATACAATTTTATTTTT |
| | | TTTAC |
| | | ATTTTTTTTCGTTGTTAATACCGTAA |
| | | (SEQ ID NO: 36) |
| Reporter | SlSMXL5-5′UTR-Stu1_R | AAGGCCTAACTTAGATACAACCCCACC |
| construction | | (SEQ ID NO: 37) |
| | SlSMXL5-5′UTR- | AAGGCCTAACTTAGATACAATTTTATT |
| | SDMStu1_R | (SEQ ID NO: 38) |
| Effector | SlJUL-Sl08g067180- | CGGGATCCATGAGCAGACCAGGAG |
| construction | BamH1_F | (SEQ ID NO: 39) |
| | SlJUL-Sl08g067180- | AAGGCCTATATGAAGACTTGTTACCAG |
| | Stu1_R | C |
| | | (SEQ ID NO: 40) |
| Genotype | SlJUL-G.T._F | ACTACTGCAAATAACAACTACCAA |
| analysis | | (SEQ ID NO: 41) |
| | SlJUL-G.T._R | GTATAATATTTGTGTAATACACGTA |
| | | (SEQ ID NO: 42) |
| Transgene | GUS_F | AACTGGACAAGGCACTAGC (SEQ ID |
| assay | | NO: 43) |
| | GUS_R | CACCGAAGTTCATGCCAGTC (SEQ ID |
| | | NO: 44) |
| | BlpR_F | TCTGCACCATCGTCAACCAC (SEQ ID |
| | | NO: 45) |
| | BlpR_R | AAACCCACGTCATGCCAGTT (SEQ ID |
| | | NO: 46) |
| Transgenic | SlJUL-VIGS-EcoR1_F | GGAATTCCCGGTGACTGGTACTGCAAT |
| plant | | (SEQ ID NO: 47) |
| | SlJUL-VIGS-Stu1_R | AAGGCCTCCAGACTTCCATCCAGAGC |
| | | G |
| | | (SEQ ID NO: 48) |
| | SlPDS-Sl03g123760-VIGS- | GGAATTCGCTGGTAGCGAATCAATG |
| | EcoR1_F | (SEQ ID NO: 49) |
| | SlPDS-Sl103g123760-VIGS- | AAGGCCTAACATCCCTTGCCTCC |
| | EcoR1_R | (SEQ ID NO: 50) |
| | SlSMXL5-Sl07g018070- | GGAATTCATTGGCTCGAGTGATCGCAA |
| | VIGS-EcoR1_F | (SEQ ID NO: 51) |
| | SlSMXL5-Sl07g018070- | TCCCCCGGGTCAGTCTTGGCCTCGTGT |
| | VIGS-Sma1_R | AC |
| | | (SEQ ID NO: 52) |
| | SlJUL-Guide2-Bbs1_TOP | ATTGCTAGCTTGAAACAAGTACAA |
| | | (SEQ ID NO: 53) |
| | SlJUL-Guide2- | AAACTTGTACTTGTTTCAAGCTAG |
| | Bbs1_BOTTOM | (SEQ ID NO: 54) |
| | SlJUL-Guide5-Bbs1_TOP | ATTGTGATTCAATCAAAATATGAG |
| | | (SEQ ID NO: 55) |
| | SlJUL-Guide5- | AAACCTCATATTTTGATTGAATCA |
| | Bbs1_BOTTOM | (SEQ ID NO: 56) |
| | SlJUL-sgRNA2_Aar1_TOP | GATTGGATGTGGTGAGCCAAGACA |
| | | (SEQ ID NO: 57) |
| | SlJUL- | AAACTGTCTTGGCTCACCACATCC |
| | sgRNA2_Aar1_BOTTOM | (SEQ ID NO: 58) |

1-3. Protoplast Preparation, Transient Expression Assay, and Immunoblotting

Fully expanded leaves of 3- to 4-week-old Arabidopsis plants were used for the protoplast isolation. Mesophyll protoplasts and plasmid DNA were prepared according to a published protocol (Hwang and Sheen, 2001). For the reporter assay, the protoplasts were diluted to a density of 2×10⁴ cells/mL and transfected with 20 μg of plasmid DNA composed of a combination of a reporter (SlSMXL5 5′UTR-GFP, SlSMXL5 5′UTR-LUC, or mSlSMXL5 5′UTR-LUC), an effector (35S:SlJUL::HA or 35S:SlJUL^{R20/81/151A}::HA), and an internal control (35S:Renilla for luciferase assay). The transfected protoplasts were incubated at room temperature for 6 hours. For a reporter assay, the relative activity of each gene was measured using a dual luciferase assay with a firefly luciferase assay system (Promega) and a Renilla luciferase assay system (Promega).

To detect the target protein levels, the total protein was extracted using a protein extraction buffer (50 mM Tris-HCl (pH 7.5), 100 mM NaCl, 5 mM EDTA, 1 mM dithiothreitol, 1×protease inhibitor cocktail (Roche), and 1% Triton X-100). Subsequently, the extracted proteins were separated using SDS-PAGE on 8 to 10% polyacrylamide gels, transferred to a nitrocellulose membrane, and then immunodetected using anti-HA (for detecting SlJUL::HA; 1:2000; Roche) or anti-GFP (for detecting SlSMXL5 5′UTR-GFP; 1:2000; Santa Cruz). The levels of the Rubisco large subunit (RbcL) were used as the control.

1-4. Confocal Microscopic Analysis

To determine the subcellular localization of SlJUL and SlJUL^{R20/81/151A}, their coding sequences were cloned into a vector containing the 35S promoter to prepare the 35S:SlJUL-GFP and 35S:SlJUL^{R20/81/151A}-GFP constructs, respectively, which were transiently expressed in protoplasts. The fluorescent GFP signals were visualized and photographed under a confocal laser scanning microscope (LSM 800; Carl Zeiss). Fluorescence signals of nuclei stained with chlorophyll and 4′,6-diamidino-2-phenylindole (DAPI) were used to determine the cytoplasmic or nuclear localization of target proteins, respectively. Chlorophyll was excited with a 640 nm wavelength laser and the emission spectrum was observed between 650 and 700 nm. For the DAPI fluorescence detection in the protoplasts, samples were treated with DAPI at a concentration of 10 μM for 10 minutes, and an excitation wavelength of 405 nm and an emission wavelength of 420 to 470 nm were used.

1-5. Histochemical Staining (GUS)

Images of GUS-stained tissues and organs were captured using a digital camera mounted on an Axioplan 2 microscope (Carl Zeiss) or Stemi SV 11 Apo stereoscope (Carl Zeiss).

1-6. Histological Embedding, Sectioning, and Imaging

The peduncle, petiole, and anther samples were fixed in FAA fixative (3.7% formaldehyde, 5% acetic acid, and 50% ethanol) at 4° C. for 16 hours, dehydrated, and embedded in paraffin wax (Paraplast; Leica Microsystems). The fixed samples were sliced into 5 μm thin sections using a Leica RM2265 microtome (Leica Biosystems). The sections were mounted onto poly-1-lysine-coated slides and stained with 0.1% safranin O. The micrographs were captured using an Axioplan 2 microscope. Measurements and counting were performed using ImageJ software (NIH; https://image j.nih.gov/ij). The peduncle was sampled at 30-days-post-anthesis (dpa) of the first raceme when the peduncle has completed its vascular development. The petioles correspond to the source leaf equivalent to the first raceme.

1-7. Virus-Induced Gene Silencing

pTRV2-derived recombinant constructs were transformed into the A. tumefaciens strain GV3101. A. tumefaciens containing pTRV1 (pYL192; Addgene plasmid # 148968; http://n2t.net/addgene:148968) or pTRV2 constructs was incubated overnight at 28° C. (OD₆₀₀=0.6), harvested, and resuspended in 10 mM MES (pH 5.5). Agrobacterium virulence was induced by adding 100 μM acetosyringone to the culture suspension and incubating at room temperature for 3 hours. A. tumefaciens cells (OD₆₀₀=1.0) containing pTRV1 or pTRV2 were mixed at a 1:1 ratio and infiltrated into the leaves of 3-week-old tomato plants. Depending on the nature of the phenotypic or anatomical recordings, the experiments were performed up to 6 weeks after Agrobacterium inoculation (30 dpa). To rule out the possible effects of TRV infection, the target gene-silenced plants were compared with plants co-inoculated with pTRV-GFP and pTRV1 as vector control. As a positive control for the VIGS experiment, the silencing effects on the PHYTOENE DESATURASE (SlPDS) gene (pTRV-PDS) were monitored.

1-8. qRT-PCR

Total RNA from the peduncles or leaves of 60-day-old plants was isolated using TRIzol™ reagent (Thermo Fisher Scientific), according to the manufacturer's instructions. Reverse transcription was carried out using 1 μg of total RNA, oligo(dT) primers, and ImProm-II reverse transcriptase (Promega). qRT-PCR was performed according to the instructions provided for the SYBR Premix ExTaq system (Takara Bio) and the StepOnePlus Real-Time PCR system (Thermo Fisher Scientific). The expression values of GLYCERALDEHYDE PHOSPHATE DEHYDROGENASE(SlGAPDH) were used to normalize the target gene expression levels.

1-9. Phloem Transport Assay

Phloem transport was assessed in the source leaves supporting the first raceme. A small area (about 25 mm²) equidistant from the leaf margin and the midrib region was marked on the abaxial surface of fully expanded leaves. The cuticular layer was gently scrapped with a scalpel, and 10 μL of an esculin solution (5 mg/mL; Alfa Aesar) was dropped on the surface (De Moliner et al., 2018; Knox et al., 2018). UV fluorescence indicating esculin transport was recorded at 0 and 10 min after esculin treatment using a Davinch-Gel imaging system MC-2000 (Davinch-K) under 306-nm UV light conditions. The extent of esculin transport was quantified in terms of relative pixel intensity using ImageJ software.

1-10. Chlorophyll Fluorescence Measurements

The photosynthetic efficiency of dark-adapted leaves from plants at 30 dpa was measured using an IMAGING-PAM chlorophyll fluorometer (MAXI Version; Walz). One measurement per plant was taken on young fully expanded leaves supporting the first raceme. Areas of interest with a diameter of 0.5 cm were randomly selected for recording data.

1-11. Measurement of Leaf CO₂ Assimilation Rate

The instantaneous values of net CO₂ assimilation rate (μmols⁻¹m⁻²) in the source leaf were determined with an LI-6400 infrared gas analyzer (LI-COR). Measurement per plant was taken on young fully expanded leaves supporting the first raceme, and five to six different plants were used. The conditions in the measuring chamber were controlled at a flow rate of 500 mols⁻¹, a saturated PAR of 1200 μmols⁻¹m⁻², 400 μmolmol⁻¹ CO₂, and a leaf temperature of 24° C.

1-12. Plant Phenotyping

The lengths and diameters of the peduncle and stem were manually quantified when at least half of the flowers were open in the inflorescences. The sizes (diameter) and weights of fruits were measured at the red ripe stage, and the first raceme was used below for measuring peduncle length. The diameters were measured with an electronic digital caliper (Mitutoyo), and the peduncle lengths were measured using 30- and 60-cm standard rulers. The fresh weight of the fruits was recorded using a digital scale (CAS), and the number of leaves, flowers, and fruits were counted in different genotypes of the same developmental age. The total fresh weight of plant roots was measured after removing the soil and foreign matter surrounding the roots and removing water, and the total dry weight was recorded using a digital scale (CAS) after drying. The individual numbers quantified are indicated for each value.

Experimental Example 2: Experimental Results

2-1. Confirmation that Genetic Function of JUL1 is Conserved in Tomato and Expressed in Vascular Tissue

(1) Confirmation of SlJUL, Orthologue of AtJUL1

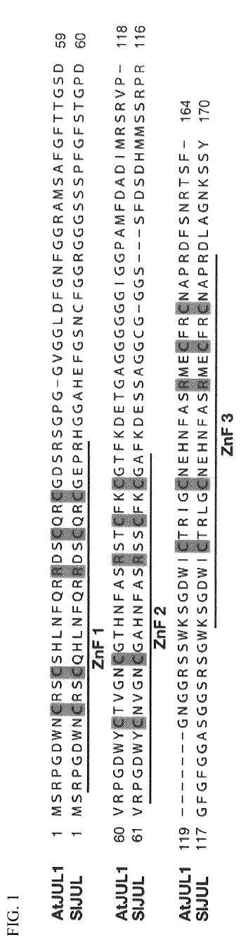

To analyze phloem development in tomato, an orthologue of AtJUL1 known as a negative regulator of phloem development was searched for. The search identified the Solyc08g067180.3.1 (SlJUL) gene encoding a protein that shares 65% (116/178) of the same amino acids with an Arabidopsis orthologue and three RanBP2-type Zinc finger (ZnF) domains. Each domain contains a conserved arginine residue (R20, R81, and R151 in ZnF1, ZnF2, and ZnF3, respectively), which is required for RNA binding (see FIG. 1).

In previous studies, it was demonstrated that AtJUL1 binds to the G-quadruplex in the 5′ UTR region of SUPPRESSOR OF MAX2 1-LIKE 5 (AtSMXL5), preventing the translation of AtSMXL5 transcripts on translationally active ribosomes and thus preventing the biosynthesis of AtSMXL5 protein, and Solyc07g018070.3.1 (SlSMXL5) was identified as an orthologue of AtSMXL5. Thereafter, as a result of calculation using a scoring algorithm that predicts the G-score based on the number of G-tetrads and the length of loops connecting the G-tetrads, the G-quadruplex in AtSMXL5 5′ UTR has a score of 41, and the SlSMXL5 5′UTR has a score of 39. This means that the 5′ UTR of SlSMXL5 may also form a G-quadruplex.

(2) Confirmation that SlJUL Suppresses Translation of SlSMXL5

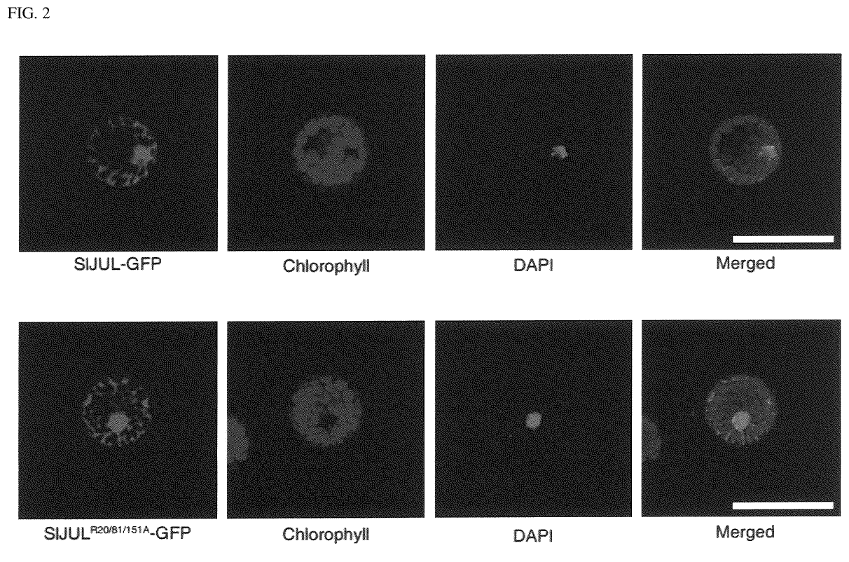

As a result of confirming the subcellular localization of SlJUL, it was found that SlJUL is located in both the cytoplasm and the nucleus (see FIG. 2). This means that SlJUL can bind to RNA to prevent the target transcript from being translated on translationally active ribosomes. To verify this, it was confirmed whether binding of SlJUL to the 5′UTR G-quadruplex of SlSMXL5 affected translation.

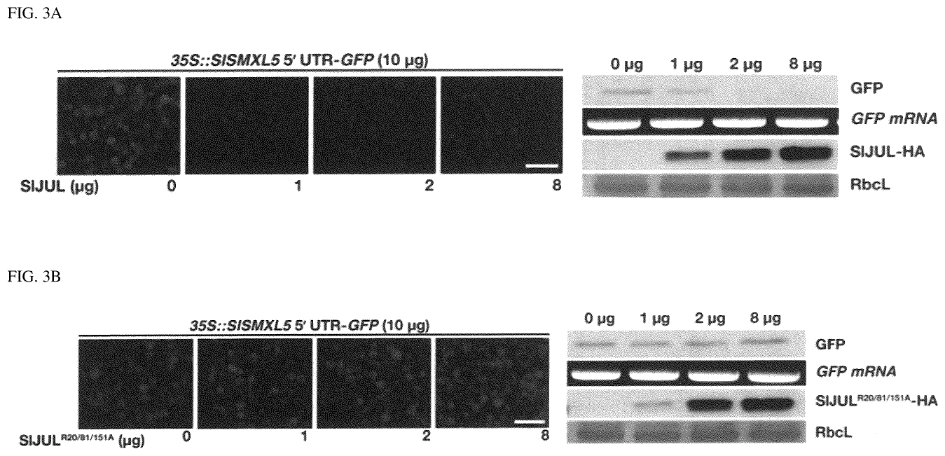

First, the protoplasts were co-transfected with a reporter SlSMXL5 5′UTR fused upstream to the GFP gene and with SlJUL as an effector. As a result of measuring GFP signal and mRNA levels after transfection, the GFP signal was reduced by the addition of the SlJUL effector in a dose-dependent manner, but there was no change in the level of GFP mRNA (see FIG. 3A).

Thereafter, to demonstrate the RNA binding activity of SlJUL to its target SlSMXL5, the conserved arginine in SlJUL was mutated to alanine to prepare SlJUL^{R20/81/151A}. As a result of measuring GFP signals by co-transfecting protoplasts with SlJUL^{R20/81/151A} and SlSMXL5 5′UTR-fused GFP, GFP signals similar to those of protoplasts transfected with SlSMXL5 5′UTR-fused GFP alone were measured independently of the concentration of SlJUL^{R20/81/151A} (see FIG. 3B).

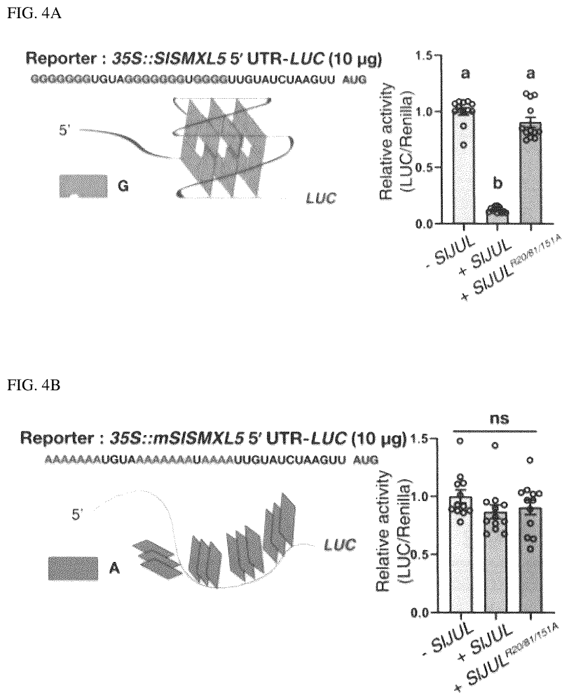

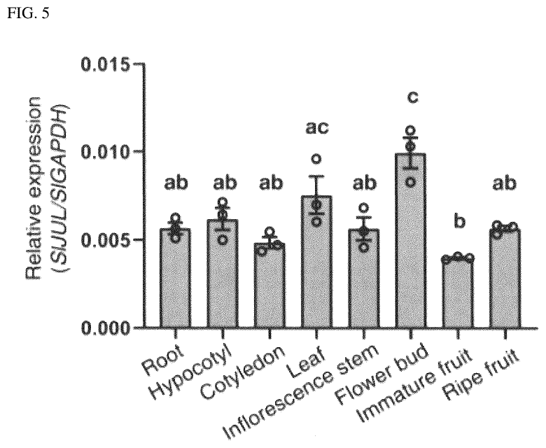

Similar to the results, it was confirmed that the protoplasts transfected with the SlSMXL5 5′UTR-fused luciferase (LUC) reporter showed a SlJUL-dependent decrease (see FIG. 4A), whereas an effector (mSlSMXL5 5′UTR) or SlJUL^{R20/81/151A} effector mutated to prevent the formation of G-quadruplex in SlSMXL5 5′UTR failed to suppress target translation (see FIG. 4B).

Through the experiments, it was verified that the interaction of SlJUL with RNA G-quadruplex motifs and the presence of a complete G-quadruplex in the SlSMXL5 5′UTR are essential for SlJUL-dependent suppression of SlSMXL5 translation.

(3) Spatial Pattern Analysis of SlJUL Expression

To further analyze the function of SIJUL, the spatial pattern of SlJUL expression in different organs spanning the early to late developmental stages was analyzed.

As a result of analyzing the expression profiles using quantitative RT-PCR, it was confirmed that the SlJUL transcript was shown to be ubiquitously present in the root, hypocotyl, cotyledons, leaf, stem, flower bud, and fruits, and the transcript was most abundant in the flowers (see FIG. 5).

Thereafter, histochemical GUS staining was performed by preparing transformed tomato plants expressing the GUS reporter gene under the control of the SlJUL promoter.

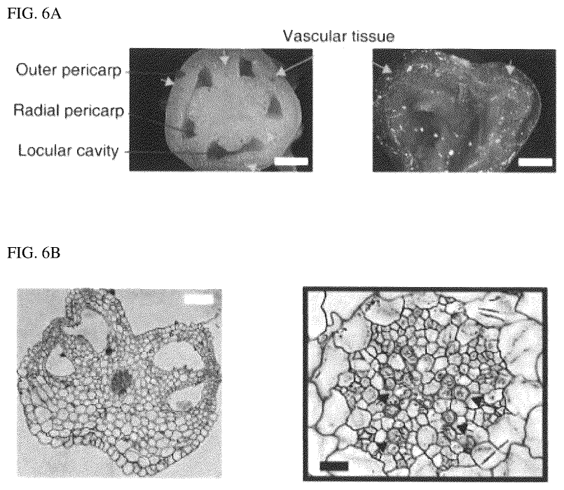

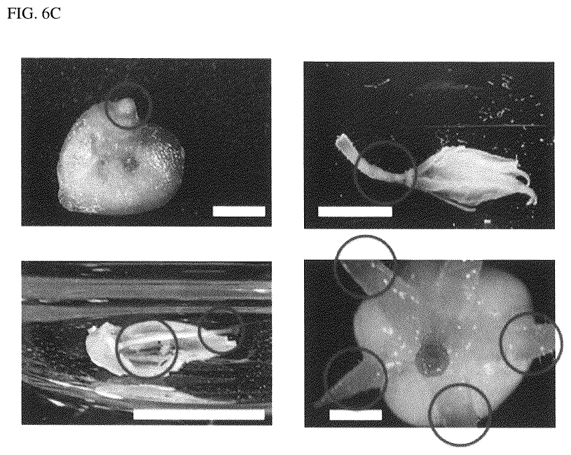

As a result of staining, GUS signals were observed in immature green fruit, red ripe fruit, and the vascular bundle structure of the anther (see FIGS. 6A and 6B), and observed in all organs including small pedicels, stamens, styles, sepals, and fruits at the embryonic root and later developmental stages of germinated seeds (see FIG. 6C).

Through the experiments, it was verified that SlJUL is ubiquitously observed in various plant organs.

2-2. Confirmation of Negative Regulation Effect of SlJUL on Tomato Phloem Differentiation

(1) Confirmation that when Expression of SlJUL is Suppressed, Tomato Phloem Differentiation is Increased

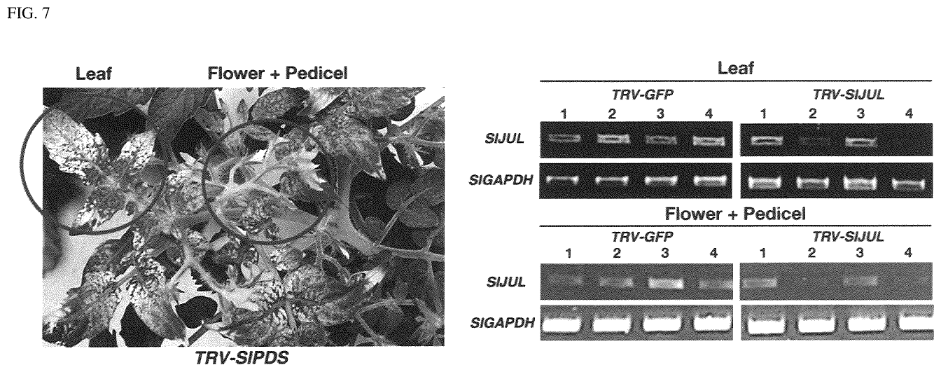

In order to confirm the effect of SlJUL on the regulation of phloem development, TRV-SlJUL with SlJUL knockdown was prepared using virus-induced genetic silencing (VIGS) technology, and these vascular bundle structures were compared with control tomato plants [TRV-SlPDS(PHYTOENE DESATURASE) and TRV-GFP] (see FIG. 7).

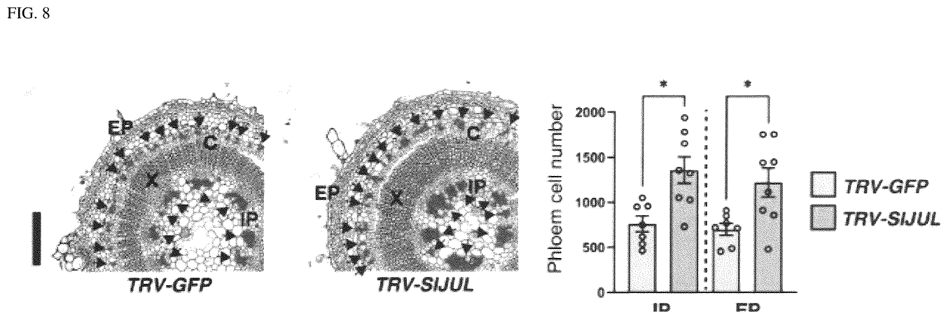

As a result of comparing peduncle cross-sections of control and SlJUL knockdown plants, it was confirmed that the suppression of SlJUL increased the total phloem cell number by approximately 1.77-fold compared to TRV-GFP plants (see FIG. 8).

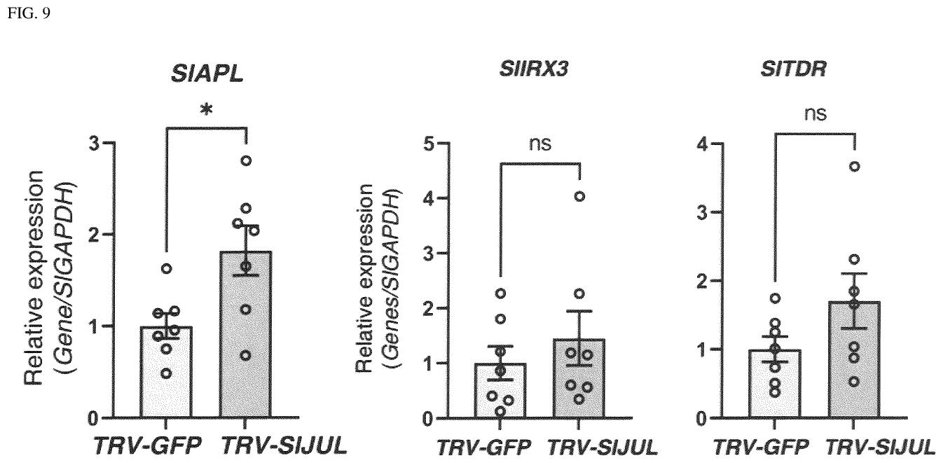

Similarly, the expression of ALTERED PHLOEM DEVELOPMENT(SlAPL), a phloem marker gene, increased about 1.82-fold in TRV-SlJUL, whereas the expression of a cambium marker gene (TDIF RECEPTOR(TDR)) and a xylem marker gene (IRREGULAR XYLEM 3(IRX3)) was not changed (see FIG. 9).

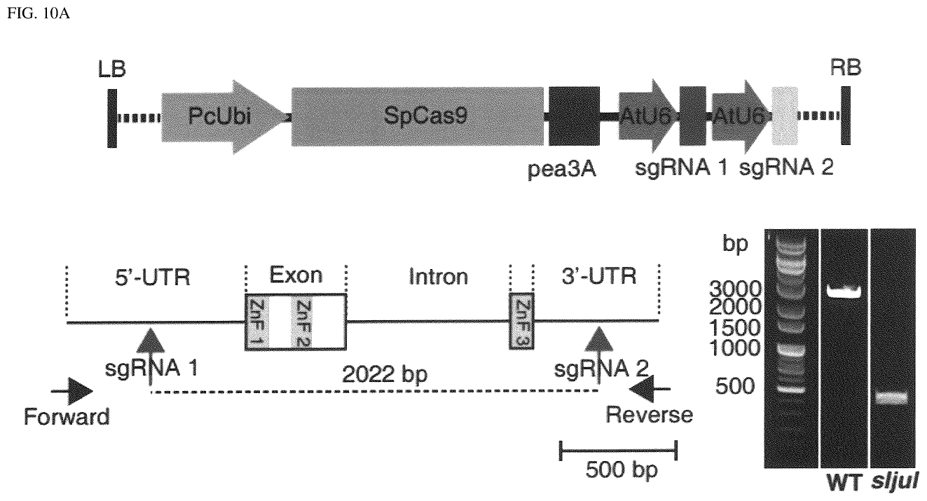

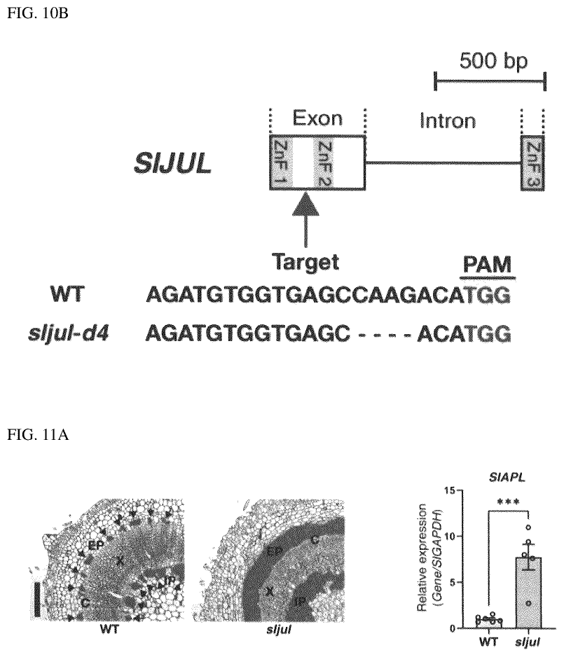

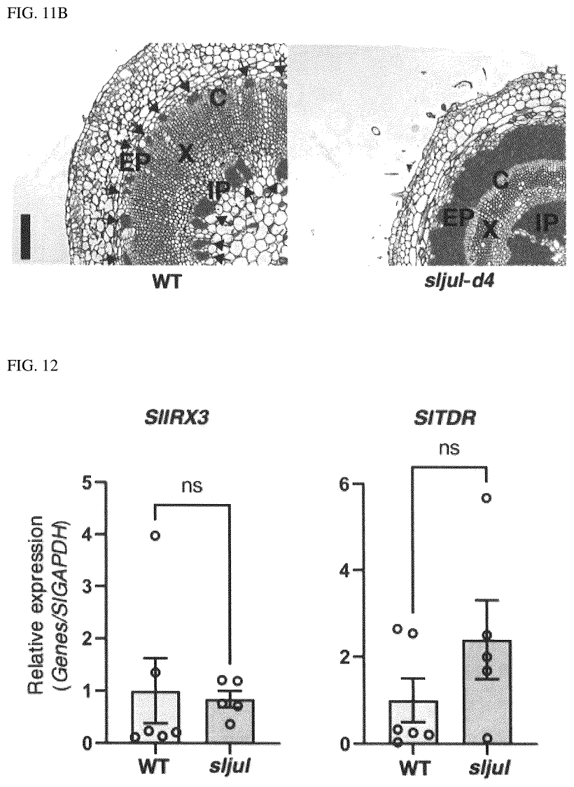

Additionally, two stable sljul null mutant lines (sljul and sljul-d4) were prepared using the CRISPR-Cas9 system (see FIGS. 10A and 10B). Similar to the TRV-SlJUL knockdown plants, the transgenic plant containing the sljul null allele showed an about 7.74-fold increase in SlAPL marker expression compared to the wild type, confirming that the phloem tissue differentiated dramatically (see FIGS. 11A and 11B). In contrast, the expression of the cambium marker gene TDR and the xylem marker gene IRX3 was not changed (see FIG. 12).

Through the experiments, it was verified that SlJUL is an evolutionarily conserved negative regulator in tomato phloem differentiation, and that the suppression of SlJUL expression can induce differentiation of phloem tissue.

(2) Confirmation that when RNA-Binding Activity of SlJUL is Suppressed, Tomato Phloem Differentiation is Increased

To confirm whether the RNA-binding activity of SlJUL is required for induction of phloem development, tomato plants expressing the mutant SlJUL^{R20/81/151A} (35S:SlJUL^{R20/81/151A}) were prepared.

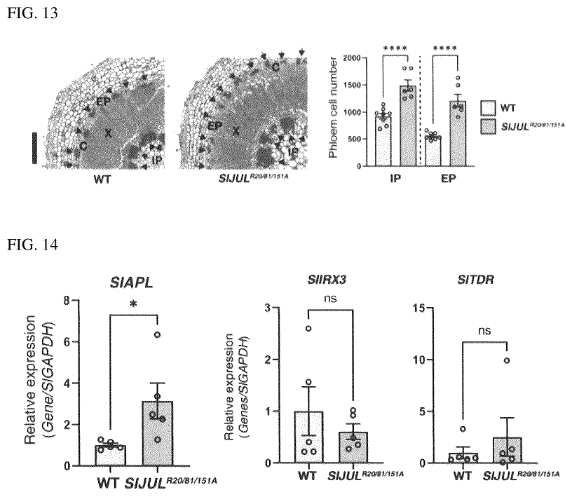

As a result of measuring the number of phloem cells and the expression of SlAPL in the transformed tomato plants expressing SlJUL^{R20/81/151A}, it was confirmed that the number of phloem cells increased about 1.84-fold compared to the wild-type control (see FIG. 13), and the expression of SlAPL increased about 3.14-fold, but the expression of the cambium marker gene TDR and the xylem marker gene IRX3 was not changed (see FIG. 14). The experimental results may mean that SlJUL^{R20/81/151A} functions as a dominant-negative form of SlJUL, competing with wild-type SlJUL when binding to the target G-quadruplex of a plant.

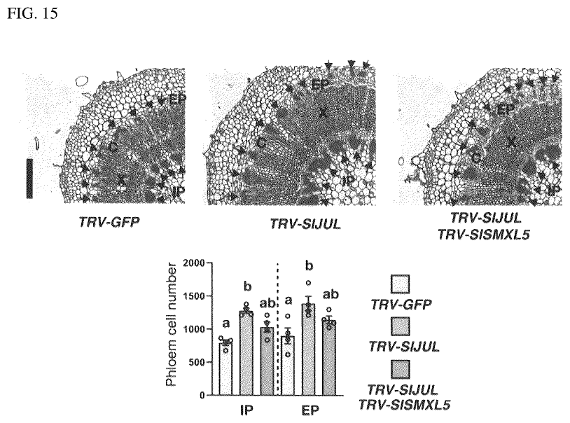

In addition, VIGS was used in SlJUL knockdown plants to prepare plants in which the expression of SlSMXL5, a target of SlJUL in phloem development, was suppressed. As a result of measuring the number of phloem cells of the plant, it was confirmed that the number of phloem cells decreased compared to the TRV-SlJUL tomato, but the number of phloem cells increased compared to the positive control (see FIG. 15).

Through the experiments, it was verified that the SlJUL-SlSMXL5 regulatory module, in which SlJUL regulates SlSMXL5, regulates the phloem differentiation of plant bodies.

2-3. Confirmation that the Suppression Level of SlJUL Determines Growth Attributes of Plant

To confirm whether anatomical changes in plant vascular bundle structure due to the suppression of SlJUL determine plant morphology or growth attributes, various growth parameters were measured in a knockdown plant (TRV-SlJUL), a suppressed plant (35S:SlJUL^{R20/81/151A}) and a knockout plant (sljul-Cas9).

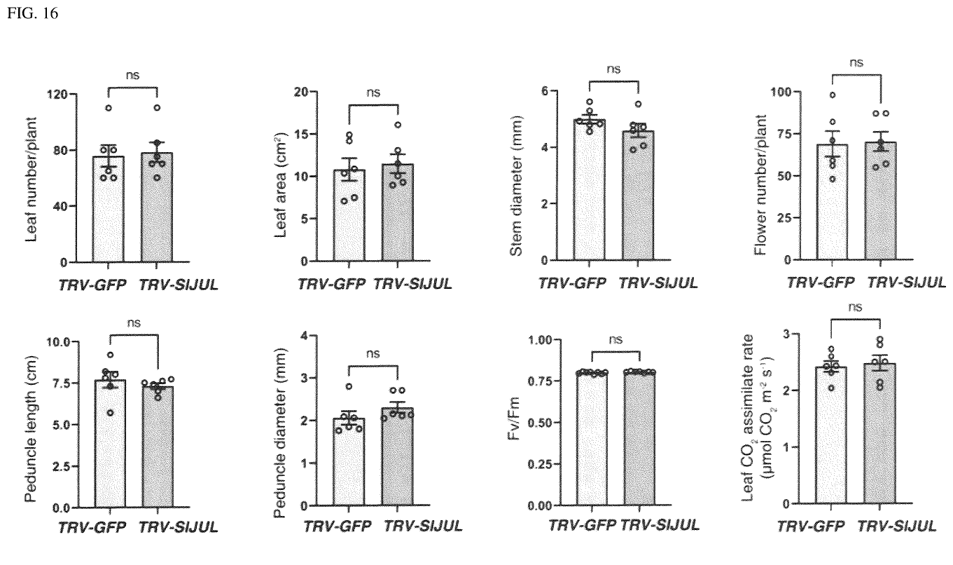

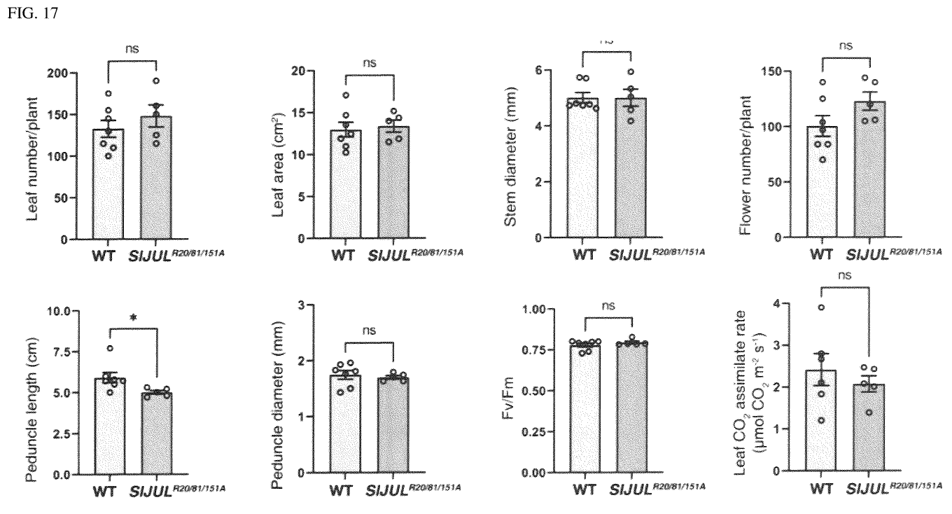

As a result of the measurement, in the case of TRV-SlJUL, no significant changes were seen in the number of leaves, leaf area, stem diameter, number of flowers, peduncle length, peduncle diameter, leaf photosynthetic efficiency and CO₂ assimilation rate compared to the control plant (see FIG. 16). In the case of 35S:SlJUL^{R20/81/151A}, no significant changes were observed in number of leaves, leaf area, stem diameter, number of flowers, peduncle diameter, leaf photosynthetic efficiency and CO₂ assimilation rate compared to the control plant, but the peduncle length, which is a measure of the phloem pathway between the source and the sink, was shown to be decreased compared to the control plant (see FIG. 17). In contrast, in the case of sljul-Cas9, peduncle diameter, leaf photosynthetic efficiency, and CO₂ assimilation rate did not change significantly compared to the control plant, but the number of leaves, leaf area, stem diameter, the number of flowers, and peduncle length were greatly reduced compared to the control plant (see FIG. 18). The unchanged CO₂ assimilation rate despite the reduced number of photosynthetic organs means a general decrease in net carbon assimilation in sljul-Cas9 plants.

In the experiment, the plant growth attributes of knockdown plants with optimally increased phloem tissue and knockout plants with a large increase in phloem tissue were compared, and through this, it was confirmed that hyperplasia of phloem can limit the development of tomatoes.

2-4. Confirmation of Transport Capacity Improvement by Increasing Phloem Tissue

(1) Confirmation that Esculin Transport Increases from SlJUL Knockdown and Knockout Plants

In order to confirm that phloem transport capacity is enhanced when the number of phloem cells increases, the transport characteristics of source leaves supplying photoassimilates to a fruit truss in SlJUL knockdown and knockout plants were analyzed. For analysis, transport was observed using the UV fluorescent dye esculin. Since esculin is loaded into the phloem by the SUCROSE TRANSPORTER (SUT) family as a sucrose analogue, it can be used to track phloem transport.

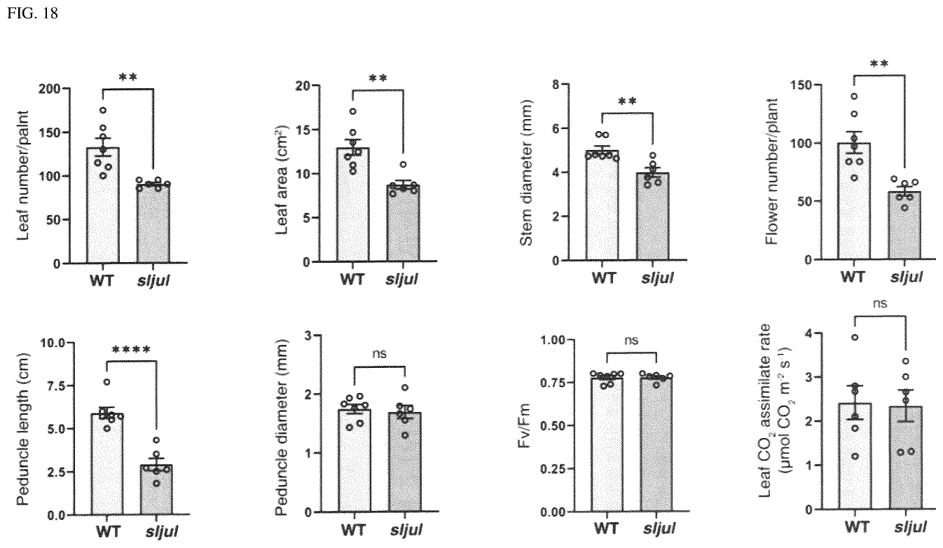

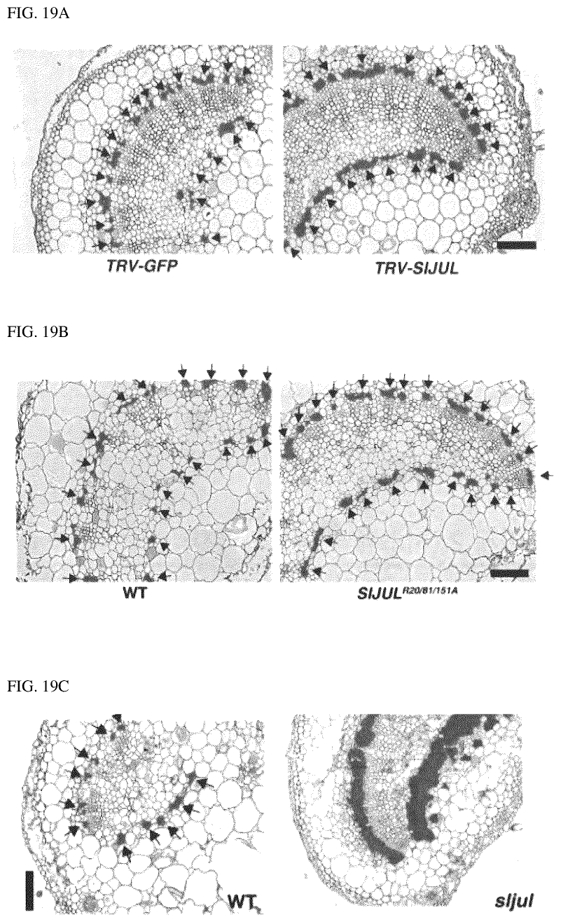

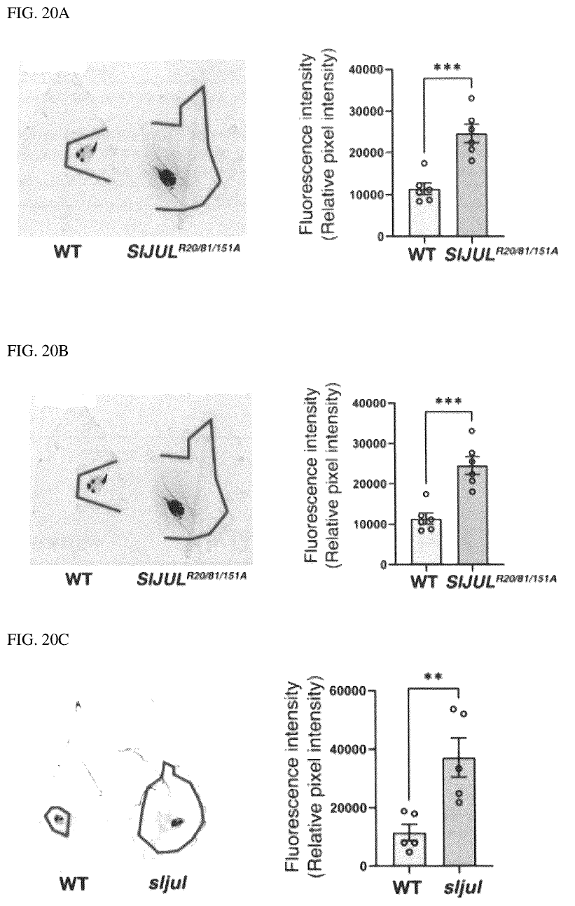

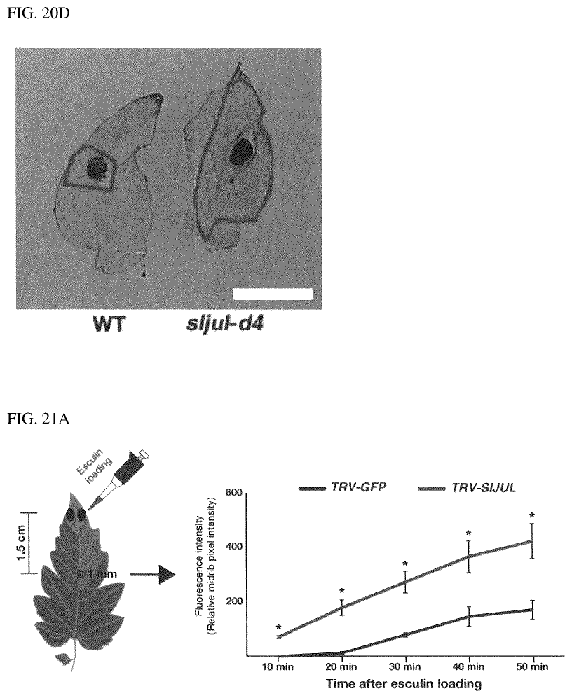

As a result, the number of phloem cells was increased in petioles of source leaves of knockdown and knockout tomato plants (see FIGS. 19A to 19C), and esculin load was increased in leaf vasculature compared to the control (see FIGS. 20A to 20D).

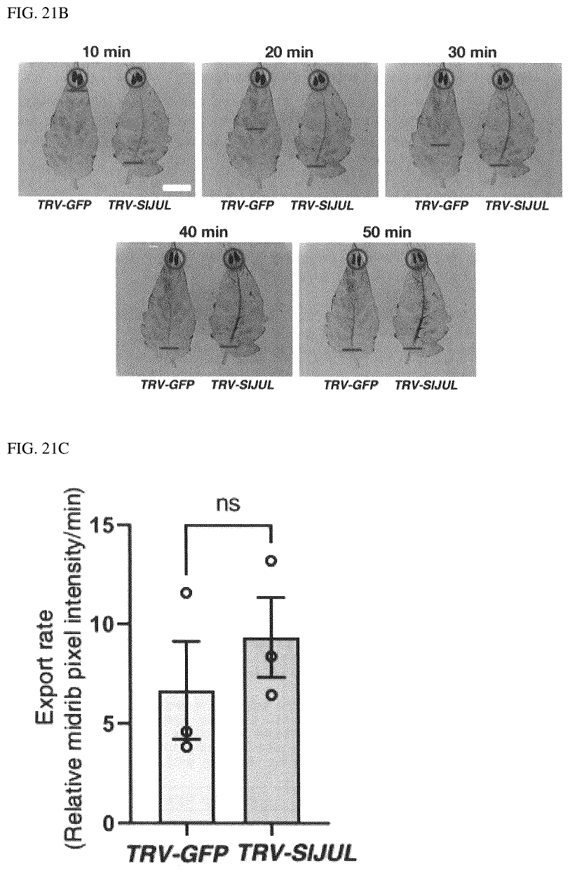

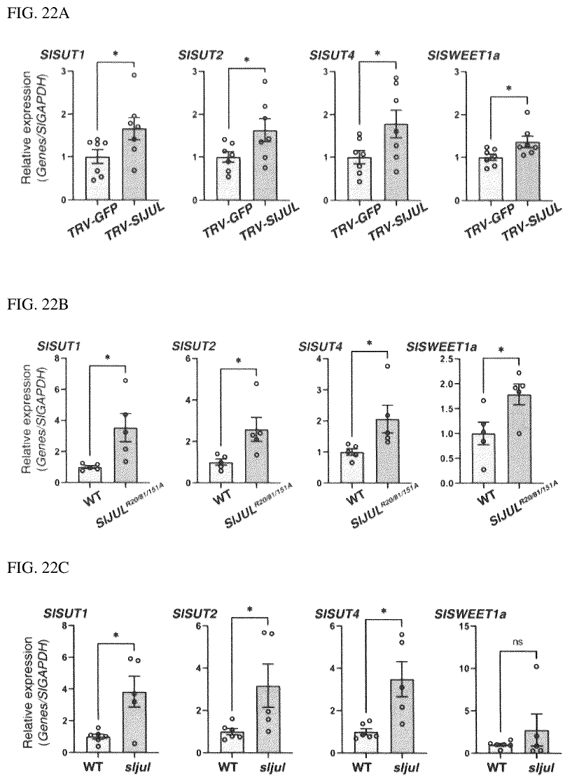

Thereafter, the phloem transport velocity and export rate of esculin were measured in the leaf midribs. As a result of the measurement, esculin reached the basal part of the midrib within 10 minutes in TRV-SlJUL leaves, but it took about 35 minutes in TRV-GFP leaves (see FIGS. 21A and 21B). However, the export rate was not significantly different in TRV-SlJUL compared to the control (see FIG. 21C). This may mean that the increased esculin transport in TRV-SlJUL was due to increased phloem loading that occurred within the first 10 minutes.

(2) Confirmation that Expression of Sucrose Transport Genes Increases from SlJUL Knockdown and Knockout Plants

The loading mechanism of activated phloem includes a sugar carrier. Specifically, sugar carriers such as the SUT and SUGARS WILL EVENTUALLY BE EXPORTED TRANSPORTERS (SWEET) families play an important role in exporting photosynthetically fixed carbon from source leaves and bringing sucrose into the phloem or into storage tissues such as fruits.

Therefore, the transcription levels of major genes involved in sucrose transport from source leaves were measured using qRT-PCR.

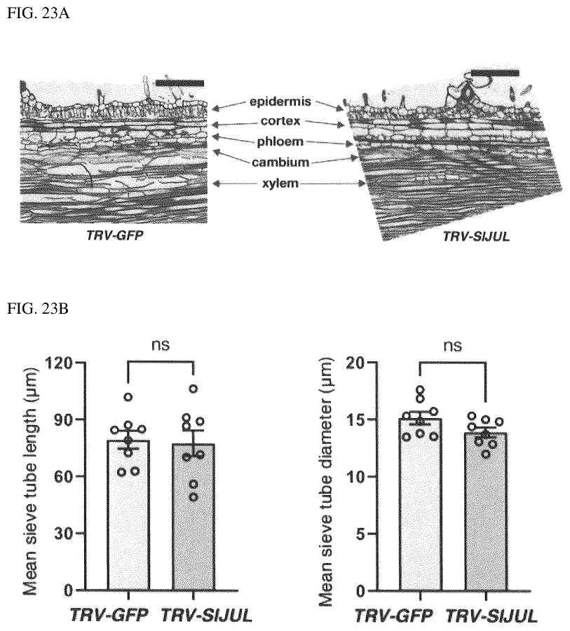

As a result, in the case of TRV-SlJUL, the expression of SlSUT1, SlSUT2, SlSUT4 and SlSWEET1a increased 1.66-fold, 1.62-fold, 1.78-fold and 1.36-fold compared to the control (see FIG. 22A), in the case of 35S:SlJUL^{R20/81/151A}, the expression of SlSUT1 increased 3.54-fold compared to the control (see FIG. 22B), and in the case of sljul-Cas9, the expression of SlSUT1 increased 3.83-fold compared to the control (see FIG. 22C).

Furthermore, since long-distance transport is affected by the length and radius of the sieve element, such features were measured in the longitudinal cross-sections of TRV-SlJUL and TRV-GFP plants, but there were no measurable differences in the sieve tubes of both plants (see FIGS. 23A and 23B).

Through the experiments, it was verified that the phloem cell population increased in SlJUL knockdown or knockout plants increases phloem transport capacity.

2-5. Confirmation that the Phloem Threshold Determines the Sink Strength of Tomato Fruits

To confirm whether increased phloem flow affected yield, average fruit number per plant, fruit size, and fruit weight were measured.

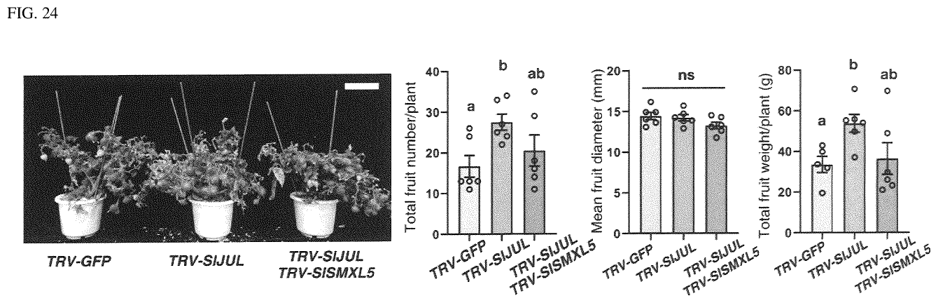

As a result of the measurements, the TRV-SlJUL knockdown plants showed no significant difference in fruit size compared to TRV-GFP, but the number of fruits increased by 37%, and the total fruit weight increased by about 60% due to the increase in the number of fruits (see FIG. 24).

Additionally, to confirm whether SlJUL regulates SlSMXL5 to increase fruit yield, TRV-SlSMXL5/TRV-SlJUL plants, in which SlSMXL5 was suppressed, were prepared using TRV-SlSMXL5 in TRV-SlJUL plants, and then the fruit yield was measured.

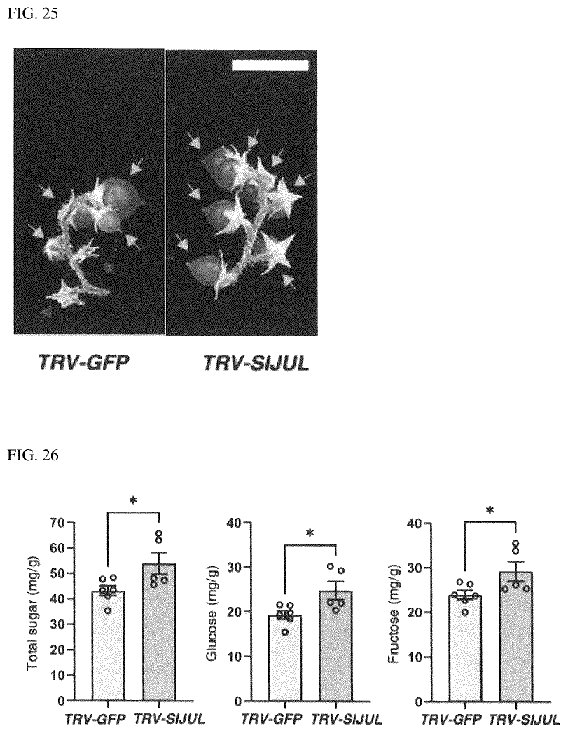

As a result of the measurement, the fruit yield of TRV-SlSMXL5/TRV-SlJUL in which SlSMXL5 was suppressed was measured at a level similar to that of the TRV-GFP control plant (see FIG. 24). The marked increase in fruit number in TRV-SlJUL plants compared to TRV-GFP plants as described above may be due to a decrease in the abortive flower/fruit ratio in TRV-SlJUL plants (see FIG. 25), and such a phenomenon is interpreted to be due to an increase in photoassimilate allocation ratio in the inflorescence sink of SlJUL-knockdown plants.

Further, it was confirmed that the total sugar content of TRV-SlJUL fruits increased by up to 25% compared to TRV-GFP fruits. Specifically, TRV-SlJUL fruits had 28% and 22% higher glucose and fructose contents than TRV-GFP fruits, respectively (see FIG. 26).

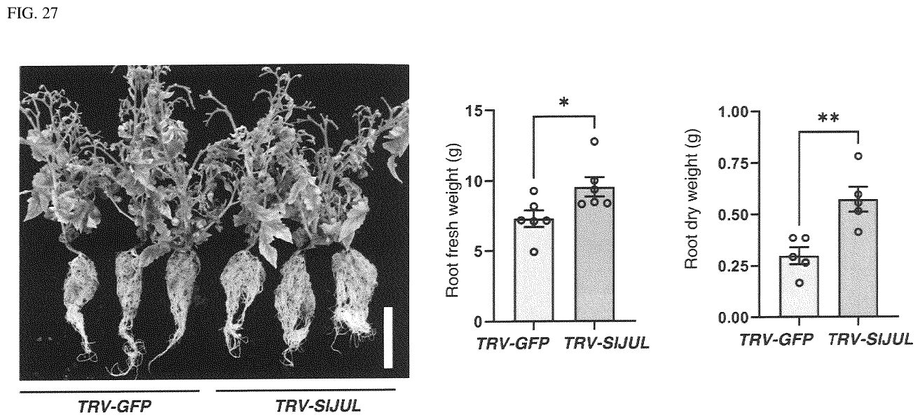

In addition, it was confirmed that the total fresh weight and dry weight of TRV-SlJUL plant roots were significantly increased compared to TRV-GFP roots (see FIG. 27).

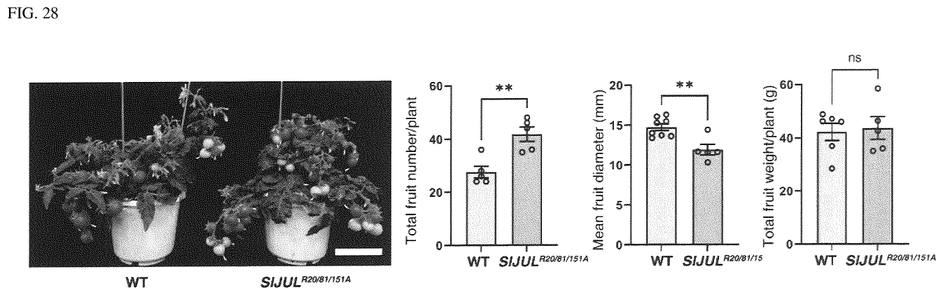

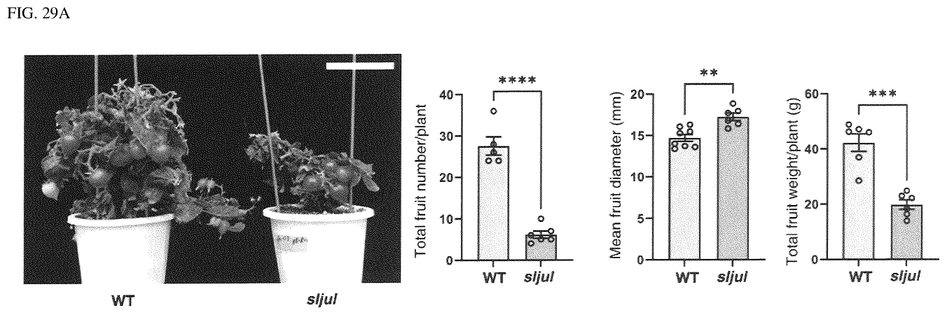

Meanwhile, in the case of 35S:SlJUL^{R20/81/151A} plants, the fruit number increased by 51% compared to the control, but the total fruit weight did not differ significantly from the control because the fruit size was smaller than that of the control (see FIG. 28).

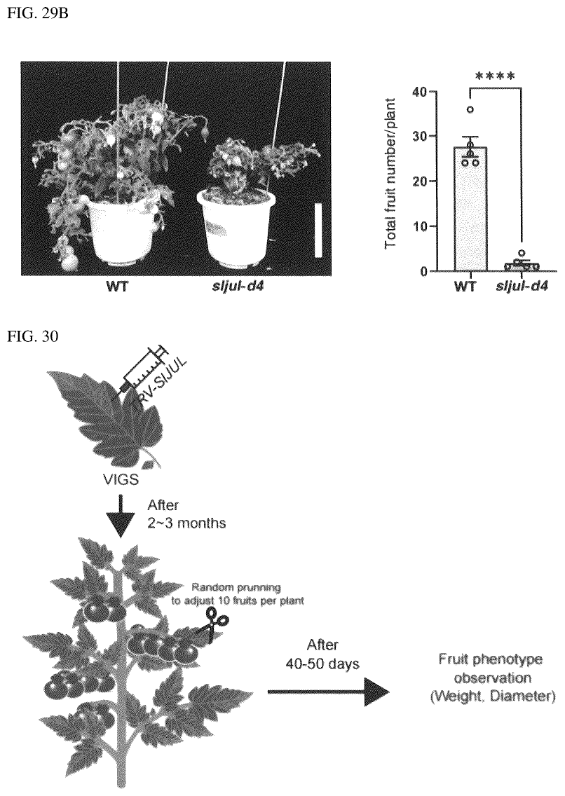

In the case of sljul-Cas9 knockout plants, unlike the knockdown plants, plant growth was weakened, the number of flowers was reduced, and the number of fruits was significantly reduced compared to the control, but the size of the fruits was larger than that of the control (FIGS. 29A and 29B). This means that there is a trade-off between fruit number and fruit size according to the increased sink competition for photoassimilate.

In addition, in order to confirm the degree of energy distribution to the sink when sufficient resources were given in tomatoes with increased phloem, tomato plants were pruned to establish fruit growth conditions under which competition was reduced and fruit growth was not impaired. The number of fruits per plant was adjusted to 10 for both TRV-SlJUL and control TRV-GFP plants, and fruit size and weight were measured as indices of sink biomass (see FIG. 30).

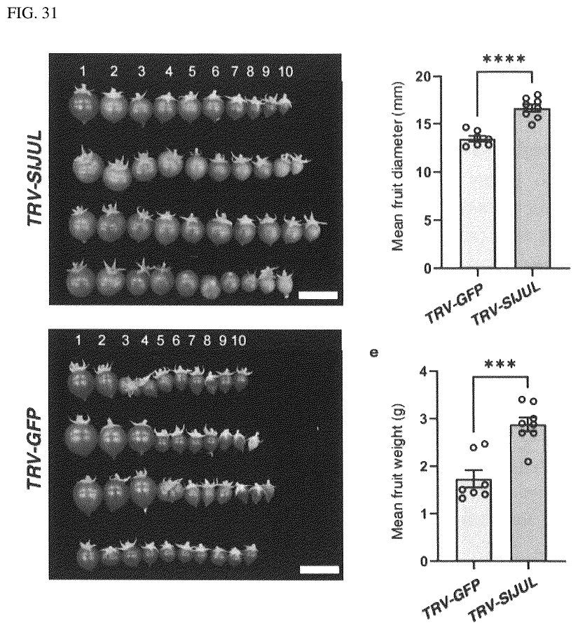

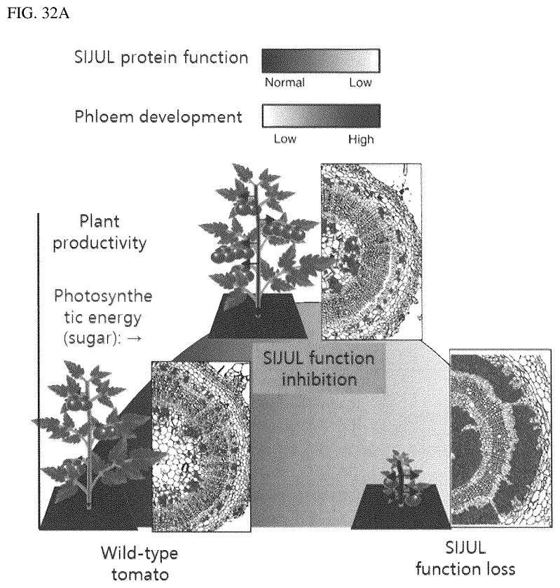

As a result of the measurements, the fruit size and weight of the TRV-SlJUL plants were significantly increased by up to 24% and 66% compared to the control fruit (see FIG. 31). This means that TRV-SlJUL fruits or sljul-Cas9 rare fruits remaining after pruning can accumulate more biomass.

Through the experiments, it was confirmed that when SlJUL expression is suppressed, the phloem cell population increases and the storage ability of tomato is improved, and there is a correlation between the increase in phloem and the increase in transport capacity. Furthermore, it was verified that fruit is more limitedly affected by resource supply than storage ability.

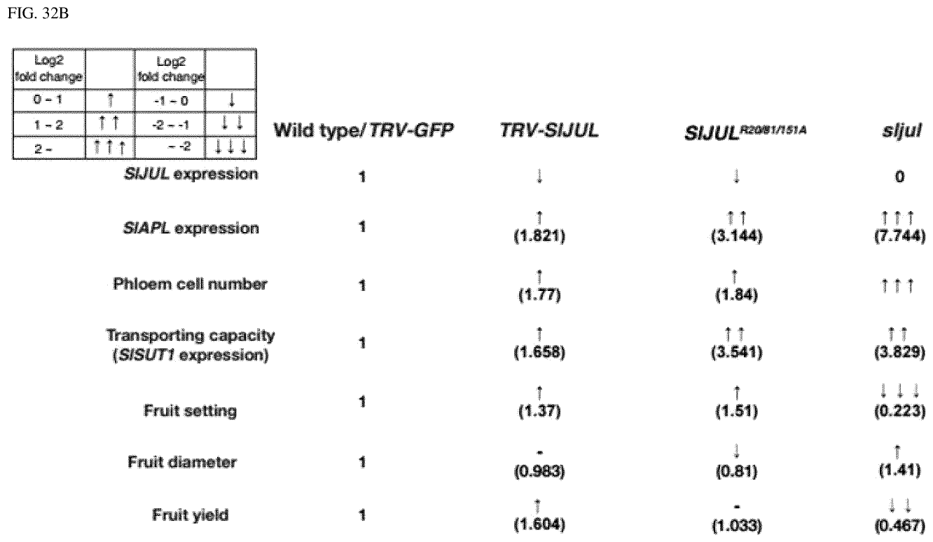

As a result of summarizing the comprehensive comparison results for phloem cell number, phloem marker gene expression, transport capacity and fruit yield in a knockdown plant (TRV-SlJUL), a dominant-negative functional plant (35S:SlJUL^{R20/81/151A}) and a knockout plant (sljul-Cas9) confirmed through the examples, it was confirmed that TRV-SlJUL plants exhibited the highest fruit yield (see FIG. 32A and 32B). Therefore, the SlJUL gene knockdown plant body TRV-SlJUL can be usefully used as a tomato cultivar with increased fruit yield.

The composition for enhancing the nutrient sink strength of the nutrient sink tissue of a plant provided by the present invention can increase the number of phloem cells and phloem transport velocity by inhibiting and suppressing the expression of the SlJUL protein or the gene encoding the SlJUL protein. Therefore, the present invention can be usefully used to increase the productivity and yield of agricultural crops.

What is claimed is:

1. A composition for enhancing the sink strength of a sink tissue of a plant, containing an expression inhibitor of an SlJUL protein or a gene encoding the SlJUL protein.

2. The composition of claim 1, wherein the expression inhibitor is one or more selected from the group consisting of (a) a virus-induced gene silencing (VIGS) vector containing an SlJUL protein or a gene encoding the SlJUL protein;
(b) a vector containing an SlJUL mutant protein or SlJUL mutant gene; and
(c) a CRISPR/Cas9 vector that edits an SlJUL protein or a gene encoding the SlJUL protein.

3. The composition of claim 2, wherein the expression inhibitor is a VIGS vector containing an SlJUL protein or a gene encoding the SlJUL protein.

4. The composition of claim 1, wherein the SlJUL protein binds to the 5′untranslated region (UTR) of SUPPRESSOR OF MAX2 1-LIKES (SlSMXL5) mRNA to suppress the expression of SlSMXL5.

5. The composition of claim 4, wherein the SlJUL protein binds to the 5UTR of the SlSMXL5 mRNA to form an RNA G-quadruplex.

6. The composition of claim 1, wherein the composition increases the number of phloem cells in a plant.

7. The composition of claim 1, wherein the composition increases the expression of an SlAPL gene.

8. The composition of claim 1, wherein the composition increases the phloem transport velocity of a plant.

9. The composition of claim 1, wherein the composition increases the expression of one or more genes selected from the group consisting of SlAPL, SlSUT2, SlSUT4, SlSUT4 and SlSWEET1a.

10. The composition of claim 1, wherein the composition increases the fruit yield of a plant.

11. The composition of claim 1, wherein the composition increases the fruit sugar content of a plant.

12. The composition of claim 1, wherein the composition increases the total fresh weight and dry weight of plant roots.

13. The composition of claim 1, wherein the plant is selected from the group consisting of food crops comprising rice, wheat, barley, corn, soybean, potato, red bean, oats, and sorghum;
vegetable crops comprising Arabidopsis thaliana, Chinese cabbage, radish, chili pepper, strawberry, tomato, watermelon, cucumber, cabbage, Korean melon, pumpkin, green onion, onion, and carrot;
industrial crops comprising ginseng, tobacco, cotton, sesame, sugar cane, sugar beet, perilla, peanut, and rapeseed;
fruit trees comprising apple, pear, jujube, peach, kiwi fruit, grape, citrus fruit, persimmon, plum, apricot, and banana trees;
root and tuber crops comprising sweet potato, Jerusalem artichoke, cassava, and yacon;
flowers comprising roses, gladiolus, gerbera, carnations, chrysanthemum, lilies, and tulips; and
fodder crops comprising ryegrass, red clover, orchard grass, alfalfa, tall fescue, and perennial ryegrass.

14. A method for enhancing the sink strength of a sink tissue of a plant, the method comprising treating a plant body with the composition of claim 1.

15. A plant body with enhanced sink strength of a sink tissue of a plant using the method of claim 14.
